(19) United States
(12) Patent Application Publication
(10) Pub. No.: US 2024/0353416 A1
(43) Pub. Date: Oct. 24, 2024

(54) ARTIFICIAL PROTEINS FOR DISPLAYING EPITOPES

(71) Applicant: Nautilus Subsidiary, Inc., Seattle, WA (US)

(72) Inventor: Terren R. CHANG, Redwood City, CA (US)

(21) Appl. No.: 18/634,901

(22) Filed: Apr. 13, 2024

Related U.S. Application Data
(60) Provisional application No. 63/495,886, filed on Apr. 13, 2023.

(51) Int. Cl. G01N 33/68

(52) U.S. Cl. CPC G01N 33/6845

(57) ABSTRACT

Provided herein is a protein including an epitope display motif, the motif having a sequence of amino acids that forms the following sequence of secondary structures: alpha₁-X₁-beta₁-X₂-beta₂-X₃-alpha₂-X₄-beta₃-X₅-beta₄, wherein “alpha” is a sequence of amino acids that forms, or is capable of forming, an alpha helix, wherein “beta” is a sequence of amino acids that forms, or is capable of forming, a beta strand, and wherein X₁, X₂, X₃, X₄ and X₅ each, independently, include a sequence of amino acids that forms an unstructured loop. Optionally, the unstructured loops can each, independently, include 2 to 10 amino acids.

### CROSS-REFERENCE TO RELATED APPLICATIONS

This application claims priority to U.S. Provisional Application No. 63/495,886, filed on Apr. 13, 2023, which is incorporated herein by reference in its entirety.

### SEQUENCE LISTING

The instant application contains a Sequence Listing which has been submitted electronically in XML format and is hereby incorporated by reference in its entirety. The XML copy, created on Apr. 10, 2024, is named “SL_50109_4022WO_US.xml” and is 92,859 bytes in size.

### BACKGROUND

The proteome is a dynamic and valuable source of biological insight and clinical diagnosis. Despite the wealth of insights gained from genomics and transcriptomics studies, which are now routine in biomedical research, a large gap remains between data on the genome/transcriptome and knowledge of how that translates into actionable phenotypes. Proteomics is crucial to bridging this gap since the proteins that constitute the proteome are the main structural and functional components that drive an individual's phenotype. Technologies for identifying and characterizing proteins at scales that match the complexity of a typical proteome lag behind DNA sequencing technologies. This is due, at least in part, to the increased variability of biochemical properties for proteins compared to DNA, as well as the significantly larger dynamic range in the quantities of different proteins present in a cell at any given time compared to DNA or RNA in the same cell. Moreover, a substantial number of the proteins predicted to comprise the human proteome have not been confidently observed to date.

Recently, binding assays have been designed for identifying large sets of polypeptides, for example, at proteome scale. See for example, U.S. Pat. Nos. 10,473,654 or 11,282,585; U.S. patent application Ser. No. 18/045,036; or Egertson et al., BioRxiv (2021), DOI: 10.1101/2021.10.11.463967, each of which is incorporated herein by reference. Increasing the number and variety of available affinity reagents can improve the range of questions that are addressable using such assays. Thus, there exists a need for reagents to facilitate production and characterization of a wide variety of affinity reagents. The present disclosure satisfies this need and provides other advantages as well.

### SUMMARY

The present disclosure provides a protein which includes an epitope display motif, the motif having a sequence of amino acids that forms the following sequence of secondary structures: alpha₁-X₁-beta₁-X₂-beta₂-X₃-alpha₂-X₄-beta₃-X₅-beta₄, wherein “alpha” is a sequence of amino acids that forms, or is capable of forming, an alpha helix, wherein “beta” is a sequence of amino acids that forms, or is capable of forming, a beta strand, and wherein X₁, X₂, X₃, X₄ and X₅ each, independently, include a sequence of amino acids that forms an unstructured loop. Optionally, the unstructured loops can each, independently, include 2 to 10 amino acids.

In particular configurations, an epitope display protein can include an amino acid sequence that is at least 75% identical to the sequence of EDP1; wherein X₁, X₂, X₃, X₄ and X₅ each include at least 2 amino acids and at most 10 amino acids. Optionally, the protein can have an amino acid sequence that is at least 80%, 85%, 90%, 95%, 96%, 97%, 98% or 99% identical to the amino acid sequence of EDP1. Further optionally the protein has amino acid sequence of EDP1. One or more of X₁, X₂, X₃, X₄ and X₅ can include a target epitope. The target epitope can include a sequence of at least 2, 3, 4, 5, 6, 7, 8, 9 or 10 amino acids. Alternatively or additionally, the target epitope can include a sequence of at most 10, 9, 8, 7, 6, 5, 4, 3 or 2 amino acids.

In particular configurations, an epitope display protein can include an amino acid sequence that is at least 75% identical to EDP2; wherein X₁, X₂, X₃, X₄, X₅, X₆, X₇, X₈, X₉, and X₁₀ each include at least 2 amino acids and at most 10 amino acids. Optionally, the protein can have amino acid sequence that is at least 80%, 85%, 90%, 95%, 96%, 97%, 98% or 99% identical to the amino acid sequence of EDP2. Further optionally the protein has the amino acid sequence of EDP2. One or more of X₁, X₂, X₃, X₄, X₅, X₆, X₇, X₈, X₉, and X₁₀ can include a target epitope. The target epitope can include a sequence of at least 2, 3, 4, 5, 6, 7, 8, 9 or 10 amino acids. Alternatively or additionally, the target epitope can include a sequence of at most 10, 9, 8, 7, 6, 5, 4, 3 or 2 amino acids.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1 shows the amino acid sequence for Peak6 (SEQ ID NO: 1) aligned with secondary structure elements including alpha helices (black bars), beta strands (grey bars) and loops (bars labeled X₁, X₂, etc.).

FIG. 2A shows an alignment of amino acid sequences for epitope display proteins GHSPG₅ (lower sequence, SEQ ID NO: 14) and pre-GHSPG₅ (upper sequence, SEQ ID NO: 15), which are in turn aligned with bars showing locations of regular secondary structure elements.

FIG. 2B shows a predicted tertiary structure for the pre-GHSPG₅ epitope display protein.

FIG. 3A shows the amino acid sequence for the EDP2-10 epitope display protein (SEQ ID NO: 53), wherein loop regions are indicated by gray shading and trimer epitopes are underlined.

FIG. 3B shows a folded structure for the EDP2-10 epitope display protein.

FIG. 3C shows the amino acid sequence for the pre-post-EDP2-10 epitope display protein (SEQ ID NO: 54), wherein the region encoding the epitope display structure motif is in bold font, the pre sequence is in regular font, the thrombin cleavage site is underlined (no italics), the post sequence is in italics, and the histidine tag is underlined and in italics.

FIG. 4A and FIG. 4B show binding data for antibodies binding to epitope display proteins.

### DETAILED DESCRIPTION

The present disclosure provides proteins configured to display epitopes for binding to affinity reagents. An epitope display protein can include a primary structure (i.e. amino acid sequence) that is capable of forming several regions of secondary structure that interact with each other to form an epitope display structure motif (i.e. the structure motif constitutes a tertiary structure). The regions of secondary structure include regions having regular secondary structure (e.g. alpha helix or beta strand) and also include loop regions that connect the regions having regular secondary structure. The loop regions typically have irregular secondary structures. In terms of tertiary structure, particularly useful loop regions are solvent exposed, being located at or near an external surface of the epitope display structure motif. As such, the regular secondary structure regions of the epitope display protein can interact in the epitope display structure motif to constrain an epitope in a loop region, thereby exposing the epitope to solvent or other molecules in the solvent. For example, an epitope that is present in a solvent exposed loop can readily bind to an affinity reagent that recognizes the epitope. An epitope display protein of the present disclosure can typically fold spontaneously to form the secondary and tertiary structures set forth herein.

Epitope display proteins of the present disclosure can be particularly useful for displaying a relatively small epitope in a way that the epitope is spatially distinct from other moieties of the protein. Thus, the epitope display structure motif can facilitate selection of affinity reagents that recognize the epitope independent of amino acids or other moieties that flank the epitope in the primary sequence of the epitope display protein. As such, an epitope display protein can be used to select an affinity reagent that is capable of recognizing a given small epitope in a variety of different sequence contexts. For example, an affinity reagent can be selected for its ability to detect a given trimer amino acid epitope in a variety of different naturally occurring proteins. Examples of relatively small epitopes include, but are not limited to, an amino acid having an added moiety (e.g. a post-translationally added moiety or an artificial moiety) or a sequence of two to eight amino acids. It will be understood that larger epitopes can also be used.

An epitope display protein can be an artificial protein, for example, having non-naturally occurring amino acid sequences in at least one, some or all of the regular secondary structures in an epitope display structure motif. In some cases, an epitope display structure motif can be derived from a de novo designed protein. Alternatively, an epitope display structure motif can be derived by modification or engineering of a naturally occurring protein structure.

A variety of different epitope display proteins can be generated from a particular epitope display structure motif. The different epitope display proteins can differ with respect to the number and/or type of epitopes present in one or more loop region of the epitope display structure motif. Nevertheless, the different epitope display proteins can share a common epitope display structure motif including, for example, some or all regular secondary structure regions in the motif, or some or all interactions between secondary structure regions of the motif (e.g. hydrogen bonding interactions that stabilize the tertiary structure of the motif). As such, an epitope display structure motif set forth herein can provide a pedestal or dais for presenting any of a variety of different epitopes to one or more affinity reagents.

Terms used herein will be understood to take on their ordinary meaning in the relevant art unless specified otherwise. Several terms used herein and their meanings are set forth below.

As used herein, the term “address” refers to a location in an array where a particular analyte (e.g. protein, or nucleic acid) is present. An address can contain a single analyte (i.e. one and only one analyte), or it can contain a population of several analytes of the same species (i.e. an ensemble of the analyte species). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁸, 1×10¹⁰, 1×10¹², 1×10¹⁴, or more addresses.

As used herein, the term “affinity agent” or “affinity reagent” refers to a molecule or other substance that is capable of specifically or reproducibly binding to an analyte (e.g. protein) or moiety (e.g. post-translational modification of a protein). An affinity agent can be larger than, smaller than or the same size as the analyte. An affinity agent may form a reversible or irreversible bond with an analyte. An affinity agent may bind with an analyte in a covalent or non-covalent manner. Affinity agents may include reactive affinity agents, catalytic affinity agents (e.g., kinases, proteases, etc.) or non-reactive affinity agents (e.g., antibodies or fragments thereof). An affinity agent can be non-reactive and non-catalytic, thereby not permanently altering the chemical structure of an analyte to which it binds. Affinity agents that can be particularly useful for binding to polypeptides include, but are not limited to, antibodies or functional fragments thereof (e.g., Fab′ fragments, F(ab′)2 fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof.

As used herein, the term “affinity tag” refers to a moiety of a molecule or other substance, the moiety being capable of specifically or reproducibly binding to a receptor. An affinity tag can be larger than, smaller than, or the same size as the receptor. An affinity tag may form a reversible or irreversible bond with a receptor. An affinity tag may bind with a receptor in a covalent or non-covalent manner. An affinity tag can include a sequence of amino acids or a sequence of nucleotides.

As used herein, the term “array” refers to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “artificial” when used in reference to a substance (e.g. protein or amino acid), means that the substance is made by human activity rather than occurring naturally. For example, a protein that is made by human activity or has a non-naturally occurring sequence of amino acids is referred to as an “artificial protein.” The term “artificial” can be used to refer to a moiety of a molecule, such that an artificial moiety is a moiety that is made by human activity and/or added to a molecule by human activity. For example, an artificial moiety can be present on an amino acid of a protein.

As used herein, the term “attached” refers to the state of two things being joined, fastened, adhered, connected or bound to each other. Attachment can be covalent or non-covalent. For example, a label can be attached to a polymer by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding affinity” or “affinity” refers to the strength or extent of binding between an affinity reagent and a binding partner. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (KA), association rate constant (kₒₙ), dissociation rate constant (k_{off}) and the like. See, for example, Segel, Enzyme Kinetics John Wiley and Sons, New York (1975), which is incorporated herein by reference in its entirety.

The term “comprising” is intended herein to be open-ended, including not only the recited elements, but further encompassing any additional elements.

As used herein, the term “conformation,” when used in reference to a protein, refers to the shape or proportionate dimensions of the protein (or portion thereof). At the molecular level conformation can be characterized by the spatial arrangement of a protein that results from the rotation of its atoms about their bonds. The conformational state of a protein can be characterized in terms of secondary structure, tertiary structure, or quaternary structure. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of a protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “fluid-phase,” when used in reference to a molecule, means the molecule is in a state wherein it is mobile in a fluid, for example, being capable of diffusing through the fluid.

As used herein, the term “exogenous,” when used in reference to a moiety of a molecule, means the moiety is not present in a natural analog of the molecule. For example, an exogenous label of an amino acid is a label that is not present on a naturally occurring amino acid. Similarly, an exogenous label that is present on an antibody is not found on the antibody in its native milieu.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “protein” refers to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; an address in an array; a tag; a label such as a luminophore; a molecular barcode such as a nucleic acid having a unique nucleotide sequence or a polypeptide having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

As used herein, the term “vessel” refers to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

The present disclosure provides proteins that can be used to display epitopes of interest. An epitope display protein can be configured to display an epitope in a loop region. Useful loop regions generally have an irregular conformation with respect to secondary structure. The peptide backbone of the amino acid residues in a loop region can include C═O moieties and N—H moieties that do not hydrogen bond to each other. As such, a loop region of an epitope display protein can accommodate a variety of different conformations, thereby making it generally well suited for substitution with any of a variety of different epitopes. Moreover, a loop region of an epitope display protein can be configured to spatially orient small epitopes (e.g. a modified amino acid or a short sequence of 2, 3, 4, 5, or 6 amino acids) away from other regions of the protein, such as regions having regular secondary structure. As such, an affinity reagent can recognize or bind to the epitope without substantial influence from other residues in the epitope display protein including, for example, residues that are adjacent to the epitope sequence in the amino acid sequence (i.e. primary structure) of the protein.

A loop region of an epitope display protein links two regions of regular secondary structure. In terms of primary and secondary structure, a loop region can occur in the linear sequence of amino acids at a region that is between two regions that form regular secondary structures. Regular secondary structures of epitope display proteins can be characterized as (i) having a sequence of consecutive residues with substantially the same phi angle (i.e. the angle of rotation about the N—Ca bond in a peptide backbone) and substantially the same psi angle (angle of rotation about the Cu—C(═O) bond in a peptide backbone), and (ii) main chain amino and carbonyl moieties that hydrogen bond to each other. Examples of regular secondary structures include alpha helices and beta strands. An alpha helix typically has (1) phi of about −60° and psi of about −50°, (2) 3.6 amino acid residues per turn, and (3) hydrogen bonds between C═O of amino acid residue n and NH of amino acid residue n+4 (this hydrogen bonding pattern does not necessarily apply to amino acid residues at the ends of an alpha helix). A beta strand typically has an extended structure with phi an psi angles in the upper left quadrant of a Ramachandran plot, and beta strands tend to be adjacent to each other in the tertiary structure of an epitope display protein, wherein C—O moieties of the backbone for one strand hydrogen bond to the N—H moieties of the backbone for an adjacent strand. Regions of regular secondary structure in an epitope display protein provide a scaffold structure that maintains the tertiary structure of the protein. Thus, loop regions that connect those regions of regular secondary structure are constrained with respect to the overall tertiary structure of the protein.

Loop regions are generally present at or near the surface of epitope display proteins. For example, the peptide backbone of the amino acid residues in a loop region can include C═O and N—H moieties that hydrogen bond to solvent or to molecules in solvent. As such, an epitope that is present in a loop region can be readily accessible to interacting with solvent or molecules in the solvent. For example, the epitope can be accessible for binding to an affinity reagent that recognizes the epitope.

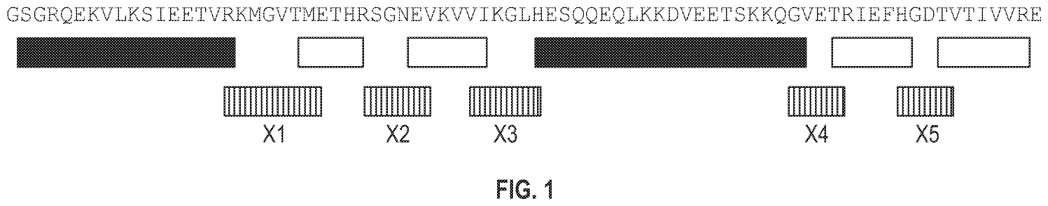

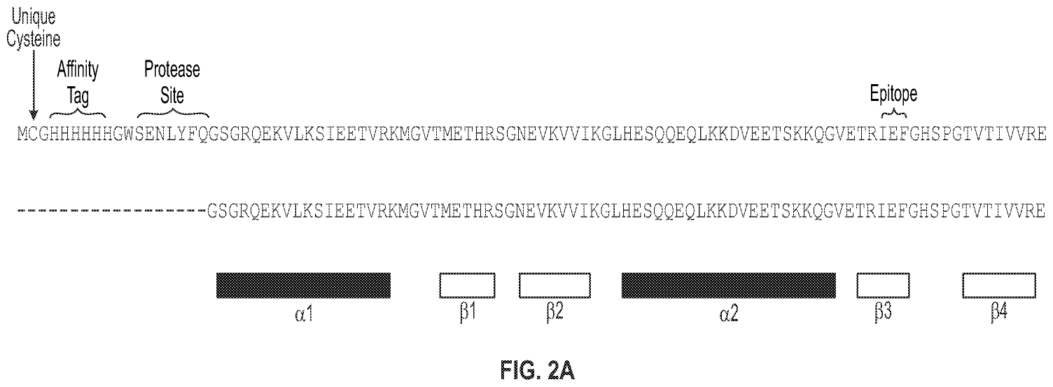

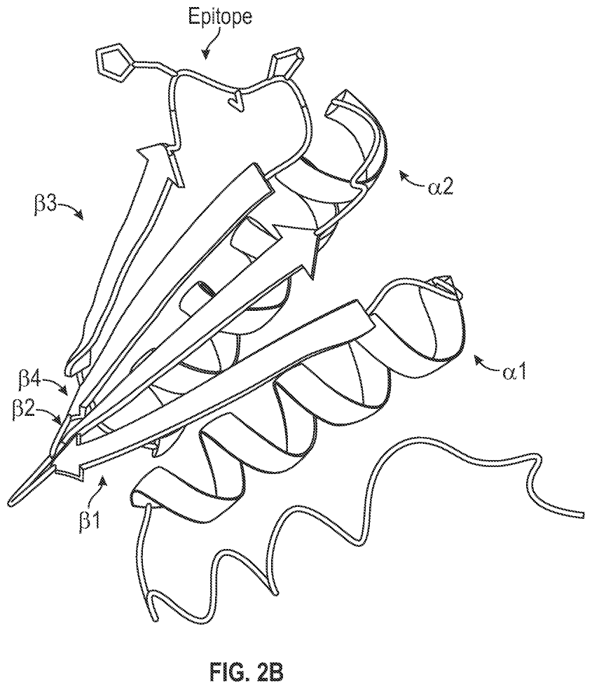

A particularly useful epitope display protein can include a motif having a secondary structure that is the same as, or similar to, those for a protein set forth herein. For example, an epitope display protein can include a motif having the following sequence of secondary structures alpha-beta₁-beta₂-alpha₂-beta₃-beta₄, wherein “alpha” indicates an alpha helix and “beta” indicates a beta strand. The regular secondary structures provide a scaffold for the motif. The motif further includes loop X₁ connecting alpha₁-beta₁, loop X₂ connecting beta₁-beta₂, loop X₃ connecting beta₂-alpha₂, loop X₄ connecting alpha₂-beta₃, and loop X₅ connecting beta₃-beta₄. Exemplary proteins having this motif include Peak6 and other proteins listed in Table 1. FIG. 1 shows the amino acid sequence for Peak6 protein aligned with secondary structure elements including alpha helices (black bars), beta strands (grey bars) and loops (bars labeled X₁, X₂, etc.). FIG. 2A shows an alignment of amino acid sequences for epitope display proteins GHSPG₅ and pre-GHSPG₅, which are in turn aligned with bars showing the regular secondary structure elements.

FIG. 2B shows a predicted tertiary structure for the pre-GHSPG₅ epitope display protein. The alpha helices and beta strands are labeled consistent with the numbering shown in FIG. 2A. The epitope, which is present in loop X₅, is labeled as well. The tertiary structure of pre-GHSPG5 includes (i) a beta sheet composed of four anti-parallel beta strands (labeled β1 through β4), (ii) a first alpha helix (labeled α1) non-covalently bonded to the beta sheet, and (iii) a second alpha helix (labeled α2) non-covalently bonded to the beta sheet. The amino acids in the first alpha helix are upstream of amino acids of the beta sheet in the amino acid sequence, the amino acids of the second alpha helix are upstream of amino acids of a first two of the beta strands (labeled β1 and β2), and the amino acids of the second alpha helix are downstream of amino acids of a second two of the beta strands (labeled β1 and β2) when their positions are considered with respect to the amino acid sequence.

A particularly useful epitope display protein can include a motif having a tertiary structure that is the same as, or similar to, those for a protein set forth herein. For example, an epitope display protein can include a tertiary structure motif that is present in GHSPG5 and pre-GHSPG₅. Optionally, an epitope display protein can include a tertiary structure motif that is present in Peak6. Similarities between protein tertiary structures can be determined using known techniques. For example, structural similarity can be determined based on a template modeling score (TM-score). See Zhang and Skolnick, Nucleic Acids Research, 33:2302-2309 (2005), which is incorporated herein by reference. An epitope display protein, or tertiary structure motif thereof, can have a TM-score of at least 0.5, 0.6, 0.7, 0.8 or 0.9 when aligned with a reference protein, or reference tertiary structure motif. The reference protein, or reference tertiary structure motif, can be a protein or motif set forth herein, for example, a protein listed in Table 1 or motif thereof. The tertiary structures can be empirically determined (e.g. via x-ray crystallography or nuclear magnetic resonance techniques) or the tertiary structures can be determined a priori (e.g. via a protein folding algorithm such as AlphaFold developed by DeepMind Ltd., London UK).

An epitope display protein of the present disclosure can be in a folded state, for example, as set forth above or elsewhere herein. Alternatively, an epitope display protein can be denatured. As such, an epitope display protein can form a molten globule or extended state. Nevertheless, a denatured epitope display protein may be considered to be capable of forming secondary or tertiary structures set forth herein when placed in a non-denaturing environment. For example, the amino acid sequence of an epitope display protein can encode a secondary or tertiary structure set forth herein. An epitope display protein can be capable of spontaneously folding into a secondary or tertiary structure set forth herein.

The present disclosure provides an epitope display protein, having an amino acid sequence that is at least 75% identical to an amino acid sequence listed in Table 1. Optionally, the epitope display protein can have an amino acid sequence that is at least 80%, 85%, 90%, 95%, 96%, 97%, 98% or 99% identical to an amino acid sequence listed in Table 1. Further optionally, an epitope display protein can have an amino acid sequence that is identical to a protein listed in Table 1. Several amino acid sequences listed in Table 1 include loop regions identified as X₁, X₂, X₃, X₄ or X₅. The loop regions can be included when determining sequence identity. For example, each of X₁, X₂, X₃, X₄ or X₅ can independently include 2, 3, 4, 5, 6, 7, 8, 9, 10 or more amino acids when determining sequence identity. Alternatively or additionally, each of X₁, X₂, X₃, X₄ or X₅ can independently include at most 10, 9, 8, 7, 6, 5, 4, 3, 2 or 1 amino acid(s) when determining sequence identity. If desired, at least one, some or all of X₁, X₂, X₃, X₄ or X₅ can be omitted when determining sequence identity.

TABLE 1
Primary Structures for Proteins Having an EDP1 Epitope Display Motif

| | (SEQ ID NO:) |
|---|---|
| Amino Acid Sequence | Name |
| GSGRQEKVLKSIEETVRKMGVTMETHRSGNEVKVVIKGLHESQ | (1) Peak6 |
| QEQLKKDVEETSKKQGVETRIEFHGDTVTIVVRE | |
| GSGRQEKVLKSIEETVX₁ETHX₂VKVVX₃ESQQEQLKKDVEETS | (2) EDP1 |
| KKQX₄RIEFXVTIVVRE | |
| MGHHHHHHGWSENLYFQGSGRQEKVLKSIEETVX₁ETHX₂VKV | (3) Pre-EDP1 |
| VX₃ESQQEQLKKDVEETSKKQX₄RIEFX₅VTIVVRE | |
| GSGRQEKVLKSIEETVX₁ETHRSGNEVKVVIKGLHESQQEQLKK | (4) EDP1X₁ |
| DVEETSKKQGVETRIEFHGDTVTIVVRE | |
| MGHHHHHHGWSENLYFQGSGRQEKVLKSIEETVX₁ETHRSGNE | (5) Pre-EDP1X₁ |
| VKVVIKGLHESQQEQLKKDVEETSKKQGVETRIEFHGDTVTIV | |
| VRE | |
| GSGRQEKVLKSIEETVRKMGVTMETHX₂VKVVIKGLHESQQEQ | (6) EDP1X₂ |
| LKKDVEETSKKQGVETRIEFHGDTVTIVVRE | |
| MGHHHHHHGWSENLYFQGSGRQEKVLKSIEETVRKMGVTME | (7) Pre-EDP1X₂ |
| THX₂VKVVIKGLHESQQEQLKKDVEETSKKQGVETRIEFHGDT | |
| VTIVVRE | |
| GSGRQEKVLKSIEETVRKMGVTMETHRSGNEVKVVX₃ESQQEQ | (8) EDP1X₃ |
| LKKDVEETSKKQGVETRIEFHGDTVTIVVRE | |
| MGHHHHHHGWSENLYFQGSGRQEKVLKSIEETVRKMGVTME | (9) Pre-EDP1X₃ |
| THRSGNEVKVVX₃ESQQEQLKKDVEETSKKQGVETRIEFHGDT | |
| VTIVVRE | |
| GSGRQEKVLKSIEETVRKMGVTMETHRSGNEVKVVIKGLHESQ | (10) EDP1X₄ |
| QEQLKKDVEETSKKQX₄RIEFHGDTVTIVVRE | |
| MGHHHHHHGWSENLYFQGSGRQEKVLKSIEETVRKMGVTME | (11) Pre-EDP1X₄ |
| THRSGNEVKVVIKGLHESQQEQLKKDVEETSKKQX₄RIEFHGDT | |
| VTIVVRE | |
| GSGRQEKVLKSIEETVRKMGVTMETHRSGNEVKVVIKGLHESQ | (12) EDP1X₅ |
| QEQLKKDVEETSKKQGVETRIEFX₅VTIVVRE | |
| MGHHHHHHGWSENLYFQGSGRQEKVLKSIEETVRKMGVTME | (13) Pre-EDP1X₅ |
| THRSGNEVKVVIKGLHESQQEQLKKDVEETSKKQGVETRIEFX₅ | |
| VTIVVRE | |
| GSGRQEKVLKSIEETVRKMGVTMETHRSGNEVKVVIKGLHESQ | (14) GHSPG₅ |
| QEQLKKDVEETSKKQGVETRIEFGHSPGTVTIVVRE | |
| MCGHHHHHHGWSENLYFQGSGRQEKVLKSIEETVRKMGVTM | (15) Pre-GHSPG₅ |
| ETHRSGNEVKVVIKGLHESQQEQLKKDVEETSKKQGVETRIEF | |
| GHSPGTVTIVVRE | |
| GSGRQEKVLKSIEETVRKMGVTMETHRSGNEVKVVIKGLHESQ | (16) GDPYG₅ |
| QEQLKKDVEETSKKQGVETRIEFGDPYGTVTIVVRE | |
| MCGHHHHHHGWSENLYFQGSGRQEKVLKSIEETVRKMGVTM | (17) Pre-GDPYG₅ |
| ETHRSGNEVKVVIKGLHESQQEQLKKDVEETSKKQGVETRIEF | |
| GDPYGTVTIVVRE | |
| GSGRQEKVLKSIEETVRKMGVTMETHRSGNEVKVVIKGLHESQ | (18) GWNKG₅ |
| QEQLKKDVEETSKKQGVETRIEFGWNKGTVTIVVRE | |
| MCGHHHHHHGWSENLYFQGSGRQEKVLKSIEETVRKMGVTM | (19) Pre-GWNKG₅ |
| ETHRSGNEVKVVIKGLHESQQEQLKKDVEETSKKQGVETRIEF | |
| GWNKGTVTIVVRE | |
| GSGRQEKVLKSIEETVRGHSPGMETHRGDPYGVKVVIGWNKG | (20) GHSPG₁- |
| HESQQEQLKKDVEETSKKQGDTRGRIEFHGDTVTIVVRE | GDPYG₂- |
| | GWNKG₃-GDTRG₄ |
| MCGHHHHHHGWSENLYFQGSGRQEKVLKSIEETVRGHSPGME | (21) Pre-GHSPG₁- |
| THRGDPYGVKVVIGWNKGHESQQEQLKKDVEETSKKQGDTRG | GDPYG₂- |
| RIEFHGDTVTIVVRE | GWNKG₃-GDTRG₄ |
| GSGRQEKVLKSIEETVRKMGDPYGTMETHRSGNEVKVVIKGLH | (22) GDPYG₁- |
| ESQQEQLKKDVEETSKKQGVETRIEFGHSPGTVTIVVRE | GHSPG₅ |
| MCGHHHHHHGWSENLYFQGSGRQEKVLKSIEETVRKMGDPY | (23) Pre-GDPYG₁- |
| GTMETHRSGNEVKVVIKGLHESQQEQLKKDVEETSKKQGVET | GHSPG₅ |
| RIEFGHSPGTVTIVVRE | |
| GSGRQEKVLKSIEETVRKMGDPYGTMETHGSLFGVKVVIKGLH | (24) GDPYG₁- |
| ESQQEQLKKDVEETSKKQGVETRIEFGHSPGTVTIVVRE | GSLFG₂-GHSPG₅ |
| MCGHHHHHHGWSENLYFQGSGRQEKVLKSIEETVRKMGDPY | (25) Pre-GDPYG₁- |
| GTMETHGSLFGVKVVIKGLHESQQEQLKKDVEETSKKQGVETR | GSLFG₂-GHSPG₅ |
| IEFGHSPGTVTIVVRE | |
| GSGRQEKVLKSIEETVRKMGDPYGTMETHGSLFGVKVVIKGLH | (26) GDPYG₁- |
| ESQQEQLKKDVEETSKKQGRDEGRIEFGHSPGTVTIVVRE | GSLFG₂-GRDEG₄- |
| | GHSPG₅ |
| MCGHHHHHHGWSENLYFQGSGRQEKVLKSIEETVRKMGDPY | (27) Pre-GDPYG₁- |
| GTMETHGSLFGVKVVIKGLHESQQEQLKKDVEETSKKQGRDE | GSLFG₂-GRDEG₄- |
| GRIEFGHSPGTVTIVVRE | GHSPG₅ |
| GSGRQEKVLKSIEETVRKMGDPYGTMETHGSLFGVKVVGDDY | (28) GDPYG₁- |
| GESQQEQLKKDVEETSKKQGRDEGRIEFGHSPGTVTIVVRE | GSLFG₂-GDDYG₃- |
| | GRDEG₄-GHSPG₅ |
| MCGHHHHHHGWSENLYFQGSGRQEKVLKSIEETVRKMGDPY | (29) Pre-GDPYG₁- |
| GTMETHGSLFGVKVVGDDYGESQQEQLKKDVEETSKKQGRDE | GSLFG₂-GDDYG₃- |
| GRIEFGHSPGTVTIVVRE | GRDEG₄-GHSPG₅ |
| GSGRQEKVLKSIEETVRKMGVTMETHRSGNEVKVVIKGLHESQ | (30) HSP_{α2} |
| QEHSPKDVEETSKKQGVETRIEFHGDTVTIVVRE | |
| MGHHHHHHGWSENLYFQGSGRQEKVLKSIEETVRKMGVTME | (31) Pre-HSP_{α2} |
| THRSGNEVKVVIKGLHESQQEHSPKDVEETSKKQGVETRIEFHG | |
| DTVTIVVRE | |

The present disclosure provides a protein, having an amino acid sequence that is at least 75% identical to GSGRQEKVLKSIEETVX₁ETHX₂VKVVX₃ESQQEQLKKDVEETSKKQX₄RIEFX₅VTIVVRE (EDP1; SEQ ID NO: 2); wherein X₁, X₂, X₃, X₄ and X₅ each include at least 2 amino acids and at most 10 amino acids. Optionally, the protein can have an amino acid sequence that is at least 80%, 85%, 90%, 95%, 96%, 97%, 98% or 99% identical to the amino acid sequence of EDP1. Further optionally the protein has amino acid sequence of EDP1.

In some configurations, a protein having the EDP1 sequence (or homologous sequence) is an epitope display protein and one or more of X₁, X₂, X₃, X₄ and X₅ includes a target epitope. Any one of X₁, X₂, X₃, X₄ or X₅ can independently include a sequence of at least 2, 3, 4, 5, 6, 7, 8, 9 or 10 amino acids. Alternatively or additionally, any one of X₁, X₂, X₃, X₄ or X₅ of a protein having the EDP1 sequence, or homologue thereof, a can independently include a sequence of at most 10, 9, 8, 7, 6, 5, 4, 3 or 2 amino acids. Exemplary target epitopes that can be included in a protein having the EDP1 sequence (or homologous sequence), such as the proteins listed in Table 1, can include, but are not limited to, HHH, HRH, YFR, WNK, FRRF (SEQ ID NO: 32), RFRF (SEQ ID NO: 33), WFR, LEEL (SEQ ID NO: 34), YWL, HFR, FST, DPY, FWR, DTR, DTV, RWWR (SEQ ID NO: 35), RDE, HSP, DPY, DTR, SLF, and DDY.

A protein having the EDP1 sequence (or homologous sequence) can have an amino acid sequence that is substantially different from the amino acid sequence GSGRQEKVLKSIEETVRKMGVTMETHRSGNEVKVVIKGLHESQQEQLKKDVEETSKKQ GVETRIEFHGDTVTIVVRE (Peak6; SEQ ID NO: 1). For example, a protein having the EDP1 sequence (or homologous sequence) can have a sequence that is at most 90%, 85%, 80%, 75%, 70% or less identical to the amino acid sequence of Peak6. Alternatively or additionally, the sequence can be at least 75%, 80%, 85%, 90%, 95%, 96%, 97%, or 98% identical to the amino acid sequence of Peak6. Comparison of amino acid sequences of Peak6 and a protein having the EDP1 sequence (or homologous sequence) can span the full sequence of the Peak6 protein or can omit sequence regions corresponding to at least one, and up to all, of the loop regions in the secondary structure of the Peak6 protein. The loop regions for Peak6 occur at amino acid residues 17-23 (loop 1), 27-31 (loop 2), 36-40 (loop 3), 59-62 (loop 4) and 67-70 (loop 5). Optionally, a comparison of amino acid sequences for Peak6 and a protein having the EDP1 sequence (or homologous sequence) can omit sequence regions corresponding to at least one, and up to all, of X₁, X₂, X₃, X₄ and X₅ of the latter.

Optionally, a protein having the EDP1 sequence (or homologous sequence) can include at least one of the following structural features: X₁ is not RKMGVTM (SEQ ID NO: 36), X₂ is not RSGNE (SEQ ID NO: 37), X₃ is not IKGLH (SEQ ID NO: 38), X₄ is not GVET (SEQ ID NO: 39), or X₅ is not HGDT (SEQ ID NO: 40). For example, the protein can include at least 1, 2, 3, 4 or 5 of the foregoing structural features. Alternatively or additionally, the protein can include at most 1, 2, 3, 4 or 5 of the foregoing structural features.

As a further option, an epitope display protein having the EDP1 epitope display structure motif can include a pre-sequence or post-sequence. The pre- or post-sequence can include, for example, a cysteine residue, an affinity tag or a protease cleavage site. The cysteine residue can be unique to the epitope display protein, for example, providing a known position for sulfur-based modification of the protein. The affinity tag can be glutathione-S-transferase or His-Tag, or any other functional affinity tag such as those set forth herein. The protease cleavage site can be a thrombin site or TEV protease site. A protease cleavage site can be positioned between the epitope display structure motif and one or both of the cysteine and affinity tag. As such protease cleavage can release one or both of the cysteine and affinity tag from the epitope display structure motif.

An epitope display protein can have an amino acid sequence that is at least 75% identical to GSGRQEKVLKSIEETVX₁ETHRSGNEVKVVIKGLHESQQEQLKKDVEETSKKQGVETRIE FHGDTVTIVVRE (EDP1X₁; SEQ ID NO: 4); wherein X₁ includes at most 10, 9, 8, 7, 6, 5, 4, 3 or 2 amino acids. Alternatively or additionally, X₁ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP1X₁. Further optionally the protein has the amino acid sequence of EDP1X₁.

An epitope display protein can have an amino acid sequence that is at least 75% identical to GSGRQEKVLKSIEETVRKMGVTMETHX₂VKVVIKGLHESQQEQLKKDVEETSKKQGVE TRIEFHGDTVTIVVRE (EDP1X₂; SEQ ID NO: 6); wherein X₂ includes at most 10, 9, 8, 7, 6, 5, 4, 3 or 2 amino acids. Alternatively or additionally, X₂ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP1X₂. Further optionally the protein has the amino acid sequence of EDP1X₂.

An epitope display protein can have an amino acid sequence that is at least 75% identical to GSGRQEKVLKSIEETVRKMGVTMETHRSGNEVKVVX₃ESQQEQLKKDVEETSKKQGVE TRIEFHGDTVTIVVRE (EDP1X₃; SEQ ID NO: 8); wherein X₃ includes at most 10, 9, 8, 7, 6, 5, 4, 3 or 2 amino acids. Alternatively or additionally, X₃ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP1X₃. Further optionally the protein has the amino acid sequence of EDP1X₃.

An epitope display protein can have an amino acid sequence that is at least 75% identical to GSGRQEKVLKSIEETVRKMGVTMETHRSGNEVKVVIKGLHESQQEQLKKDVEETSKKQ X₄RIEFHGDTVTIVVRE (EDP1X₄; SEQ ID NO: 10); wherein X₄ includes at most 10, 9, 8, 7, 6, 5, 4, 3 or 2 amino acids. Alternatively or additionally, X₄ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP1X₄. Further optionally the protein has the amino acid sequence of EDP1X₄.

An epitope display protein can have an amino acid sequence that is at least 75% identical to GSGRQEKVLKSIEETVRKMGVTMETHRSGNEVKVVIKGLHESQQEQLKKDVEETSKKQ GVETRIEFX₅VTIVVREE (EDP1X₅; SEQ ID NO: 12); wherein X₅ includes at most 10, 9, 8, 7, 6, 5, 4, 3 or 2 amino acids. Alternatively or additionally, X₅ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP1X₅. Further optionally the protein has the amino acid sequence of EDP1X₅.

For a protein having the sequence of EDP1, EDP1X₁, or homologue thereof, X₁ can include the amino acid sequence RX_{1A}, wherein X_{1A} includes a sequence of at least 2, 3, 4, 5 or 6 amino acids. Alternatively or additionally, XIA can include a sequence of at most 6, 5, 4, 3 or 2 amino acids. Optionally, X₁ can include the amino acid sequence X_{1B}M, wherein X_{1B} includes a sequence of at least 2, 3, 4, 5 or 6 amino acids. Alternatively or additionally, X₁B can include a sequence of at most 6, 5, 4, 3 or 2 amino acids. In a further option, X₁ can include the amino acid sequence RX_{1C}M, wherein X_{1C} includes a sequence of at least 2, 3, 4, or 5 amino acids. Alternatively or additionally, X_{1C} can include a sequence of at most 5, 4, 3 or 2 amino acids.

For a protein having the sequence of EDP1, EDP1X₂, or homologue thereof, X₂ can include the amino acid sequence RX_{2A}, wherein X_{2A} includes a sequence of at least 2, 3, 4, 5 or 6 amino acids. Alternatively or additionally, X_{2A} can include a sequence of at most 6, 5, 4, 3 or 2 amino acids. Optionally, X₂ can include the amino acid sequence X_{2B}E, wherein X_{2B} includes a sequence of at least 2, 3, 4, 5 or 6 amino acids. Alternatively or additionally, X_{2B} can include a sequence of at most 6, 5, 4, 3 or 2 amino acids. In a further option, X₂ can include the amino acid sequence RX_{2C}E, wherein X_{2C} includes a sequence of at least 2, 3, 4, or 5 amino acids. Alternatively or additionally, X_{2C} can include a sequence of at most 5, 4, 3 or 2 amino acids.

For a protein having the sequence of EDP1, EDP1X₃, or homologue thereof, X₃ can include the amino acid sequence IX_{3A}, wherein X_{3A} includes a sequence of at least 2, 3, 4, 5 or 6 amino acids. Alternatively or additionally, X_{3A} can include a sequence of at most 6, 5, 4, 3 or 2 amino acids. Optionally, X₃ can include the amino acid sequence X_{3B}H, wherein X_{3B} includes a sequence of at least 2, 3, 4, 5 or 6 amino acids. Alternatively or additionally, X_{3B} can include a sequence of at most 6, 5, 4, 3 or 2 amino acids. In a further option, X₃ can include the amino acid sequence IX_{3C}H, wherein X_{3C} includes a sequence of at least 2, 3, 4, or 5 amino acids. Alternatively or additionally, X_{3C} can include a sequence of at most 5, 4, 3 or 2 amino acids.

For a protein having the sequence of EDP1, EDP1X₄, or homologue thereof, X₄ can include the amino acid sequence GX_{4A}, wherein X_{4A} includes a sequence of at least 2, 3, 4, 5 or 6 amino acids. Alternatively or additionally, X_{4A} can include a sequence of at most 6, 5, 4, 3 or 2 amino acids. Optionally, X₄ can include the amino acid sequence X_{4B}T, wherein X_{4B} includes a sequence of at least 2, 3, 4, 5 or 6 amino acids. Alternatively or additionally, X_{4B} can include a sequence of at most 6, 5, 4, 3 or 2 amino acids. In a further option, X₄ can include the amino acid sequence GX_{4C}T, wherein X_{4C} includes a sequence of at least 2, 3, 4, or 5 amino acids. Alternatively or additionally, X_{4C} can include a sequence of at most 5, 4, 3 or 2 amino acids.

For a protein having the sequence of EDP1, EDP1X₅, or homologue thereof, X₅ can include the amino acid sequence HX_{5A}, wherein X_{5A} includes a sequence of at least 2, 3, 4, 5 or 6 amino acids. Alternatively or additionally, X_{5A} can include a sequence of at most 6, 5, 4, 3 or 2 amino acids. Optionally, X₅ can include the amino acid sequence X_{5B}T, wherein X_{5B} includes a sequence of at least 2, 3, 4, 5 or 6 amino acids. Alternatively or additionally, X_{5B} can include a sequence of at most 6, 5, 4, 3 or 2 amino acids. In a further option, X₅ can include the amino acid sequence HX_{5C}T, wherein X_{5C} includes a sequence of at least 2, 3, 4, or 5 amino acids. Alternatively or additionally, X_{5C} can include a sequence of at most 5, 4, 3 or 2 amino acids.

In some cases, it may be beneficial to flank an epitope with a glycine residue. A glycine residue can provide a larger range of rotation at the junction between a loop region and a region having a regular secondary structure (e.g. alpha helix or beta strand). As such, a glycine can be present at a position in the amino acid sequence of an epitope display protein that occurs between a region of regular secondary structure and an epitope. For a protein having the sequence of EDP1, EDP1X₁, or homologue thereof, X₁ can include the amino acid sequence GX_{1D}, wherein X_{1D} includes a sequence of at least 2, 3, 4, 5 or 6 amino acids. Alternatively or additionally, X_{1D} can include a sequence of at most 6, 5, 4, 3 or 2 amino acids. Optionally, X₁ can include the amino acid sequence X_{1E}G, wherein X_{1E} includes a sequence of at least 2, 3, 4, 5 or 6 amino acids. Alternatively or additionally, X_{1E} can include a sequence of at most 6, 5, 4, 3 or 2 amino acids. As a further option, X₁ can include the amino acid sequence GX_{1F}G, wherein X_{1F} includes a sequence of at least 2, 3, 4, or 5 amino acids. Alternatively or additionally, X_{1F} can include a sequence of at most 5, 4, 3 or 2 amino acids.

For a protein having the sequence of EDP1, EDP1X₂, or homologue thereof, X₂ can include the amino acid sequence GX_{2D}, wherein X_{2D} includes a sequence of at least 2, 3, 4, 5 or 6 amino acids. Alternatively or additionally, X_{2D} can include a sequence of at most 6, 5, 4, 3 or 2 amino acids. Optionally, X₂ can include the amino acid sequence X_{2E}G, wherein X_{2E} includes a sequence of at least 2, 3, 4, 5 or 6 amino acids. Alternatively or additionally, X_{2E} can include a sequence of at most 6, 5, 4, 3 or 2 amino acids. As a further option, X₂ can include the amino acid sequence GX_{2F}G, wherein X_{2F} includes a sequence of at least 2, 3, 4, or 5 amino acids. Alternatively or additionally, X_{2F} can include a sequence of at most 5, 4, 3 or 2 amino acids.

For a protein having the sequence of EDP1, EDP1X₃, or homologue thereof, X₃ can include the amino acid sequence GX_{3D}, wherein X_{3D} includes a sequence of at least 2, 3, 4, 5 or 6 amino acids. Alternatively or additionally, X_{3D} can include a sequence of at most 6, 5, 4, 3 or 2 amino acids. Optionally, X₃ can include the amino acid sequence X_{3E}G, wherein X_{3E} includes a sequence of at least 2, 3, 4, 5 or 6 amino acids. Alternatively or additionally, X_{3E} can include a sequence of at most 6, 5, 4, 3 or 2 amino acids. As a further option, X₃ can include the amino acid sequence GX_{3F}G, wherein X_{3F} includes a sequence of at least 2, 3, 4, or 5 amino acids. Alternatively or additionally, X_{3F} can include a sequence of at most 5, 4, 3 or 2 amino acids.

For a protein having the sequence of EDP1, EDP1X₄, or homologue thereof, X₄ can include the amino acid sequence GX_{4D}, wherein X_{4D} includes a sequence of at least 2, 3, 4, 5 or 6 amino acids. Alternatively or additionally, X_{4D} can include a sequence of at most 6, 5, 4, 3 or 2 amino acids. Optionally, X₄ can include the amino acid sequence X_{4E}G, wherein X_{4E} includes a sequence of at least 2, 3, 4, 5 or 6 amino acids. Alternatively or additionally, X_{4E} can include a sequence of at most 6, 5, 4, 3 or 2 amino acids. As a further option, X₄ can include the amino acid sequence GX_{4F}G, wherein X_{4F} includes a sequence of at least 2, 3, 4, or 5 amino acids. Alternatively or additionally, X_{4F} can include a sequence of at most 5, 4, 3 or 2 amino acids.

For a protein having the sequence of EDP1, EDP1X₅, or homologue thereof, X₅ can include the amino acid sequence GX_{5D}, wherein X_{5D} includes a sequence of at least 2, 3, 4, 5 or 6 amino acids. Alternatively or additionally, X_{5D} can include a sequence of at most 6, 5, 4, 3 or 2 amino acids. Optionally, X₅ can include the amino acid sequence X_{5E}G, wherein X_{5E} includes a sequence of at least 2, 3, 4, 5 or 6 amino acids. Alternatively or additionally, X_{5E} can include a sequence of at most 6, 5, 4, 3 or 2 amino acids. As a further option, X₅ can include the amino acid sequence GX_{5F}G, wherein X_{5F} includes a sequence of at least 2, 3, 4, or 5 amino acids. Alternatively or additionally, X_{5F} can include a sequence of at most 5, 4, 3 or 2 amino acids.

For a protein having the sequence of EDP1, EDP1X₁, or homologue thereof, X₁ can include any of a variety of amino acid sequences including, but not limited to, RKMGVTM (SEQ ID NO: 36), RGHSPGM (SEQ ID NO: 41), HSP, GHSPG (SEQ ID NO: 42), DPY, GDPYG (SEQ ID NO: 43), WNK or GWNKG (SEQ ID NO: 44). Optionally, X₁ can include a target epitope selected from HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

For a protein having the sequence of EDP1, EDP1X₂, or homologue thereof, X₂ can include any of a variety of amino acid sequences including, but not limited to, RSGNE, HSP, GHSPG, DPY, GDPYG, WNK or GWNKG. Optionally, X₂ can include a target epitope selected from HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

For a protein having the sequence of EDP1, EDP1X₃, or homologue thereof, X₃ can include any of a variety of amino acid sequences including, but not limited to, IKGLH, HSP, GHSPG, DPY, GDPYG, WNK or GWNKG. Optionally, X₃ can include a target epitope selected from HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

For a protein having the sequence of EDP1, EDP1X₄, or homologue thereof, X₄ can include any of a variety of amino acid sequences including, but not limited to, GVET, HSP, GHSPG, DPY, GDPYG, WNK or GWNKG. Optionally, X₄ can include a target epitope selected from HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

For a protein having the sequence of EDP1, EDP1X₅, or homologue thereof, X₅ can include any of a variety of amino acid sequences including, but not limited to, IKGLH, GHSPGT, HSP, GHSPG, DPY, GDPYG, WNK or GWNKG. Optionally, X₅ can include a target epitope selected from HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

An epitope display protein of the present disclosure can be configured to present an epitope of interest in a single loop region or in a plurality of loop regions. For example, the same epitope can be displayed in at least 2, 3, 4, 5, 6, 7, 8, 9, 10 or more loop regions of an epitope display protein. Alternatively or additionally, the same epitope can be displayed in no more than 10, 9, 8, 7, 6, 5, 4, 3, 2 or 1 loop regions of an epitope display protein. Presenting the same epitope in multiple loop regions of a protein can provide the benefit of increasing avidity of binding between the epitope display protein and an affinity reagent that recognizes the epitope. Typically, an epitope that is presented in one or more loop regions is not present in any other region of the epitope display protein. For example, the epitope may be absent in regions of the epitope display protein having regular secondary structures (e.g. alpha helices or beta strands). In other words, the epitopes may be absent from the epitope display structure motif of the epitope display protein.

In particular configurations, a protein having the EDP1 sequence (or homologous sequence), can display a given epitope of interest in two or more of X₁, X₂, X₃, X₄ and X₅. For example, a protein having the EDP1 sequence (or homologous sequence), can display the same epitope in X₁ and X₂, in X₁ and X₃, in X₁ and X₄, in X₁ and X₅, in X₂ and X₃, in X₂ and X₄, in X₂ and X₅, in X₃ and X₄, in X₃ and X₅, or in X₄ and X₅.

Optionally, a protein having the EDP1 sequence (or homologous sequence), can display a given epitope of interest in three or more of X₁, X₂, X₃, X₄ and X₅. For example, a protein having the EDP1 sequence (or homologous sequence), can display the same epitope in X₁, X₂ and X₃; in X₁, X₂ and X₄; in X₁, X₂ and X₅; in X₂, X₃ and X₄ in X₂, X₃ and X₅; in X₃, X₄ and X₅; in X₁, X₃ and X₄; in X₁, X₃ and X₅; in X₁, X₄ and X₅; or in X₂, X₄ and X₅.

Optionally, a protein having the EDP1 sequence (or homologous sequence), can display a given epitope of interest in four or more of X₁, X₂, X₃, X₄ and X₅. For example, a protein having the EDP1 sequence (or homologous sequence), can display the same epitope in X₁, X₂, X₃ and X₄; in X₁, X₃, X₄ and X₅; in X₂, X₃, X₄ and X₅; in X₁, X₂, X₄ and X₅; or in X₁, X₂, X₃ and X₅. Optionally, a protein having the EDP1 sequence (or homologous sequence), can display a given epitope of interest in all five of X₁, X₂, X₃, X₄ and X₅.

An epitope display protein of the present disclosure can be configured to present a plurality of different epitopes of interest, for example, in different loop regions, respectively. For example, different epitopes can be displayed in at least 2, 3, 4, 5, 6, 7, 8, 9, 10 or more loop regions of an epitope display protein. Alternatively or additionally, different epitopes can be displayed in no more than 10, 9, 8, 7, 6, 5, 4, 3, 2 or 1 loop regions of an epitope display protein. Presenting different epitopes in multiple loop regions of a protein can provide the benefit of increasing the variety of affinity reagents that can be bound to the protein. Typically, the different epitopes that are presented in multiple loop regions are not present in any other region of the epitope display protein. For example, the epitopes may be absent in regions of the epitope display protein having regular secondary structures (e.g. alpha helices or beta strands). In other words, the epitopes may be absent in the epitope display structure motif of the epitope display protein.

Turning to the example of a protein having the EDP1 sequence (or homologous sequence), the protein can display different epitopes of interest in two or more of X₁, X₂, X₃, X₄ and X₅. For example, a protein having the EDP1 sequence (or homologous sequence), can display different epitopes in X₁ and X₂, in X₁ and X₃, in X₁ and X₄, in X₁ and X₅, in X₂ and X₃, in X₂ and X₄, in X₂ and X₅, in X₃ and X₄, in X₃ and X₅, or in X₄ and X₅.

Optionally, a protein having the EDP1 sequence (or homologous sequence), can display different epitopes of interest in three or more of X₁, X₂, X₃, X₄ and X₅. For example, a protein having the EDP1 sequence (or homologous sequence), can display different epitope in X₁, X₂ and X₃; in X₁, X₂ and X₄; in X₁, X₂ and X₅; in X₂, X₃ and X₄ in X₂, X₃ and X₅; in X₃, X₄ and X₅; in X₁, X₃ and X₄; in X₁, X₃ and X₅; in X₁, X₄ and X₅; or in X₂, X₄ and X₅.

Optionally, a protein having the EDP1 sequence (or homologous sequence), can display different epitopes of interest in four or more of X₁, X₂, X₃, X₄ and X₅. For example, a protein having the EDP1 sequence (or homologous sequence), can display different epitopes in X₁, X₂, X₃ and X₄; in X₁, X₃, X₄ and X₅; in X₂, X₃, X₄ and X₅; in X₁, X₂, X₄ and X₅; or in X₁, X₂, X₃ and X₅. Optionally, a protein having the EDP1 sequence (or homologous sequence), can display different epitope of interest in all five of X₁, X₂, X₃, X₄ and X₅.

The present disclosure provides a protein, having an amino acid sequence that is at least 75% identical to an amino acid sequence listed in Table 2. Optionally, the protein can have an amino acid sequence that is at least 80%, 85%, 90%, 95%, 96%, 97%, 98% or 99% identical to an amino acid sequence listed in Table 2. Further optionally the protein can have an amino acid sequence of a protein listed in Table 2. Several amino acid sequences listed in Table 2 include loop regions identified as X₁, X₂, X₃, X₄, X₅, X₆, X₇, X₈, X₉ or X₁₀. The loop regions can be included when determining sequence identity. For example, each of X₁, X₂, X₃, X₄, X₅, X₆, X₇, X₈, X₉ or X₁₀ can independently include 2, 3, 4, 5, 6, 7, 8, 9, 10 or more amino acids when determining sequence identity. Alternatively or additionally, each of X₁, X₂, X₃, X₄, X₅, X₆, X₇, X₈, X₉ or X₁₀ can independently include at most 10, 9, 8, 7, 6, 5, 4, 3, 2 or 1 amino acid(s) when determining sequence identity. If desired, at least one, some or all of X₁, X₂, X₃, X₄, X₅, X₆, X₇, X₈, X₉ or X₁₀ can be omitted when determining sequence identity.

TABLE 2
Primary Structures for Proteins Having an EDP2 Epitope Display Motif

| | (SEQ ID NO:) |
|---|---|
| Amino Acid Sequence | Name |
| EESKKRQWALEDFEIGRPLGKGKFGNVYLAREKQSKFILALKV | (50) Human Aurora |
| LFKAQLEKAGVEHQLRREVEIQSHLRHPNILRLYGYFHDATRV | Kinase A |
| YLILEYAPLGTVYRELQKLSKFDEQRTATYITELANALSYCHSK | |
| RVIHRDIKPENLLLGSAGELKIADFGWSVHAPSSRRTTLCGTLD | |
| YLPPEMIEGRMHDEKVDLWSLGVLCYEFLVGKPPFEANTYQET | |
| YKRISRVEFTFPDFVTEGARDLISRLLKHNPSQRPMLREVLEHP | |
| WITANSSKPSNCQNKESASKQS | |
| MESKKRQWALEDFEIGRPLGKX₁GNVYLAREX₂ILALKVLFKAQ | (51) EDP2 |
| LEKAGVEHQLRREVEIQSHX₃NILRLYGYFHX₄RVYLILEYAPLG | |
| TVYRELQKLX₅EQRTATYITELANALSYCHSKRVIHRDIKPENLL | |
| LX₆LKIADFGWSVHAX₇LDYLPPEMIX₈EKVDLWSLGVLCYEFL | |
| VGKPPFX₉YQETYKRISX10EGARDLISRLLKHNPSQRPMLREVL | |
| EHPWITANSSKPSNAQNKESASKQS | |
| MSPILGYWKIKGLVQPTRLLLEYLEEKYEEHLYERDEGDKWRN | (52) pre-post-EDP2 |
| KKFELGLEFPNLPYYIDGDVKLTQSMAIIRYIADKHNMLGGCPK | |
| ERAEISMLEGAVLDIRYGVSRIAYSKDFETLKVDFLSKLPEMLK | |
| MFEDRLCHKTYLNGDHVTHPDFMLYDALDVVLYMDPMCLDA | |
| FPKLVCFKKRIEAIPQIDKYLKSSKYIAWPLQGWQATFGGGDHP | |
| PKSDGSTSGSGHHHHHHSAGLVPRGSTAIGMKETAAAKFERQH | |
| MDSPDLGTMESKKRQWALEDFEIGRPLGKX₁GNVYLAREX₂ILA | |
| LKVLFKAQLEKAGVEHQLRREVEIQSHX₃NILRLYGYFHX₄RVY | |
| LILEYAPLGTVYRELQKLX5EQRTATYITELANALSYCHSKRVIH | |
| RDIKPENLLLX₆LKIADFGWSVHAX₇LDYLPPEMIX₈EKVDLWSL | |
| GVLCYEFLVGKPPFX₉YQETYKRISX₁₀EGARDLISRLLKHNPSQR | |
| PMLREVLEHPWITANSSKPSNAQNKESASKQSDYKDDDDKHH | |
| HHHHHH | |
| MESKKRQWALEDFEIGRPLGKSLFGNVYLAREKDTRFILALKV | (53) EDP2-10 |
| LFKAQLEKAGVEHQLRREVEIQSHLLPQNILRLYGYFHLEFRVY | |
| LILEYAPLGTVYRELQKLHSPDEQRTATYITELANALSYCHSKR | |
| VIHRDIKPENLLLGHPDELKIADFGWSVHAPSSRRDRIAGTLDY | |
| LPPEMIEGRFSTEKVDLWSLGVLCYEFLVGKPPFFRETYQETYK | |
| RISRVEFTFPSVHTEGARDLISRLLKHNPSQRPMLREVLEHPWIT | |
| ANSSKPSNAQNKESASKQS | |
| MSPILGYWKIKGLVQPTRLLLEYLEEKYEEHLYERDEGDKWRN | (54) pre-post-EDP2-10 |
| KKFELGLEFPNLPYYIDGDVKLTQSMAIIRYIADKHNMLGGCPK | |
| ERAEISMLEGAVLDIRYGVSRIAYSKDFETLKVDFLSKLPEMLK | |
| MFEDRLCHKTYLNGDHVTHPDFMLYDALDVVLYMDPMCLDA | |
| FPKLVCFKKRIEAIPQIDKYLKSSKYIAWPLQGWQATFGGGDHP | |
| PKSDGSTSGSGHHHHHHSAGLVPRGSTAIGMKETAAAKFERQH | |
| MDSPDLGTMESKKRQWALEDFEIGRPLGKSLFGNVYLAREKDT | |
| RFILALKVLFKAQLEKAGVEHQLRREVEIQSHLLPQNILRLYGY | |
| FHLEFRVYLILEYAPLGTVYRELQKLHSPDEQRTATYITELANA | |
| LSYCHSKRVIHRDIKPENLLLGHPDELKIADFGWSVHAPSSRRD | |
| RIAGTLDYLPPEMIEGRFSTEKVDLWSLGVLCYEFLVGKPPFFR | |
| ETYQETYKRISRVEFTFPSVHTEGARDLISRLLKHNPSQRPMLRE | |
| VLEHPWITANSSKPSNAQNKESASKQSDYKDDDDKHHHHHHHH | |
| MESKKRQWALEDFEIGRPLGKX₁GNVYLAREKQSKFILALKVL | (55) EDP2X₁ |
| FKAQLEKAGVEHQLRREVEIQSHLRHPNILRLYGYFHDATRVY | |
| LILEYAPLGTVYRELQKLSKFDEQRTATYITELANALSYCHSKR | |
| VIHRDIKPENLLLGSAGELKIADFGWSVHAPSSRRTTLCGTLDY | |
| LPPEMIEGRMHDEKVDLWSLGVLCYEFLVGKPPFEANTYQETY | |
| KRISRVEFTFPDFVTEGARDLISRLLKHNPSQRPMLREVLEHPWI | |
| TANSSKPSNCQNKESASKQS | |
| MESKKRQWALEDFEIGRPLGKGKFGNVYLAREX₂ILALKVLFK | (56) EDP2X₂ |
| AQLEKAGVEHQLRREVEIQSHLRHPNILRLYGYFHDATRVYLIL | |
| EYAPLGTVYRELQKLSKFDEQRTATYITELANALSYCHSKRVIH | |
| RDIKPENLLLGSAGELKIADFGWSVHAPSSRRTTLCGTLDYLPP | |
| EMIEGRMHDEKVDLWSLGVLCYEFLVGKPPFEANTYQETYKRI | |
| SRVEFTFPDFVTEGARDLISRLLKHNPSQRPMLREVLEHPWITA | |
| NSSKPSNCQNKESASKQS | |
| MESKKRQWALEDFEIGRPLGKGKFGNVYLAREKQSKFILALKV | (57) EDP2X₃ |
| LFKAQLEKAGVEHQLRREVEIQSHX₃NILRLYGYFHDATRVYLI | |
| LEYAPLGTVYRELQKLSKFDEQRTATYITELANALSYCHSKRVI | |
| HRDIKPENLLLGSAGELKIADFGWSVHAPSSRRTTLCGTLDYLP | |
| PEMIEGRMHDEKVDLWSLGVLCYEFLVGKPPFEANTYQETYK | |
| RISRVEFTFPDFVTEGARDLISRLLKHNPSQRPMLREVLEHPWIT | |
| ANSSKPSNCQNKESASKQS | |
| MESKKRQWALEDFEIGRPLGKGKFGNVYLAREKQSKFILALKV | (58) EDP2X₄ |
| LFKAQLEKAGVEHQLRREVEIQSHLRHPNILRLYGYFHX₄RVYL | |
| ILEYAPLGTVYRELQKLSKFDEQRTATYITELANALSYCHSKRV | |
| IHRDIKPENLLLGSAGELKIADFGWSVHAPSSRRTTLCGTLDYLP | |
| PEMIEGRMHDEKVDLWSLGVLCYEFLVGKPPFEANTYQETYK | |
| RISRVEFTFPDFVTEGARDLISRLLKHNPSQRPMLREVLEHPWIT | |
| ANSSKPSNCQNKESASKQS | |
| MESKKRQWALEDFEIGRPLGKGKFGNVYLAREKQSKFILALKV | (59) EDP2X₅ |
| LFKAQLEKAGVEHQLRREVEIQSHLRHPNILRLYGYFHDATRV | |
| YLILEYAPLGTVYRELQKLX₅EQRTATYITELANALSYCHSKRVI | |
| HRDIKPENLLLGSAGELKIADFGWSVHAPSSRRTTLCGTLDYLP | |
| PEMIEGRMHDEKVDLWSLGVLCYEFLVGKPPFEANTYQETYK | |
| RISRVEFTFPDFVTEGARDLISRLLKHNPSQRPMLREVLEHPWIT | |
| ANSSKPSNCQNKESASKQS | |
| MESKKRQWALEDFEIGRPLGKGKFGNVYLAREKQSKFILALKV | (60) EDP2X₆ |
| LFKAQLEKAGVEHQLRREVEIQSHLRHPNILRLYGYFHDATRV | |
| YLILEYAPLGTVYRELQKLSKFDEQRTATYITELANALSYCHSK | |
| RVIHRDIKPENLLLX₆LKIADFGWSVHAPSSRRTTLCGTLDYLPP | |
| EMIEGRMHDEKVDLWSLGVLCYEFLVGKPPFEANTYQETYKRI | |
| SRVEFTFPDFVTEGARDLISRLLKHNPSQRPMLREVLEHPWITA | |
| NSSKPSNCQNKESASKQS | |
| MESKKRQWALEDFEIGRPLGKGKFGNVYLAREKQSKFILALKV | (61) EDP2X7 |
| LFKAQLEKAGVEHQLRREVEIQSHLRHPNILRLYGYFHDATRV | |
| YLILEYAPLGTVYRELQKLSKFDEQRTATYITELANALSYCHSK | |
| RVIHRDIKPENLLLGSAGELKIADFGWSVHAX₇LDYLPPEMIEGR | |
| MHDEKVDLWSLGVLCYEFLVGKPPFEANTYQETYKRISRVEFT | |
| FPDFVTEGARDLISRLLKHNPSQRPMLREVLEHPWITANSSKPS | |
| NCQNKESASKQS | |
| MESKKRQWALEDFEIGRPLGKGKFGNVYLAREKQSKFILALKV | (62) EDP2X₈ |
| LFKAQLEKAGVEHQLRREVEIQSHLRHPNILRLYGYFHDATRV | |
| YLILEYAPLGTVYRELQKLSKFDEQRTATYITELANALSYCHSK | |
| RVIHRDIKPENLLLGSAGELKIADFGWSVHAPSSRRTTLCGTLD | |
| YLPPEMIX₈EKVDLWSLGVLCYEFLVGKPPFEANTYQETYKRIS | |
| RVEFTFPDFVTEGARDLISRLLKHNPSQRPMLREVLEHPWITAN | |
| SSKPSNCQNKESASKQS | |
| MESKKRQWALEDFEIGRPLGKGKFGNVYLAREKQSKFILALKV | (63) EDP2X₉ |
| LFKAQLEKAGVEHQLRREVEIQSHLRHPNILRLYGYFHDATRV | |
| YLILEYAPLGTVYRELQKLSKFDEQRTATYITELANALSYCHSK | |
| RVIHRDIKPENLLLGSAGELKIADFGWSVHAPSSRRTTLCGTLD | |
| YLPPEMIEGRMHDEKVDLWSLGVLCYEFLVGKPPFX₉YQETYK | |
| RISRVEFTFPDFVTEGARDLISRLLKHNPSQRPMLREVLEHPWIT | |
| ANSSKPSNCQNKESASKQS | |
| MESKKRQWALEDFEIGRPLGKGKFGNVYLAREKQSKFILALKV | (64) EDP2X₁₀ |
| LFKAQLEKAGVEHQLRREVEIQSHLRHPNILRLYGYFHDATRV | |
| YLILEYAPLGTVYRELQKLSKFDEQRTATYITELANALSYCHSK | |
| RVIHRDIKPENLLLGSAGELKIADFGWSVHAPSSRRTTLCGTLD | |
| YLPPEMIEGRMHDEKVDLWSLGVLCYEFLVGKPPFEANTYQET | |
| YKRISX₁₀EGARDLISRLLKHNPSQRPMLREVLEHPWITANSSKP | |
| SNCQNKESASKQS | |

The present disclosure provides a protein, having an amino acid sequence that is at least 75% identical to MESKKRQWALEDFEIGRPLGKX₁GNVYLAREX₂ILALKVLFKAQLEKAGVEHQLRREVE IQSHX₃NILRLYGYFHX₄RVYLILEYAPLGTVYRELQKLX₅EQRTATYITELANALSYCHS KRVIHRDIKPENLLLX₆LKIADFGWSVHAX₇LDYLPPEMIX₈EKVDLWSLGVLCYEFLVG KPPFX₉YQETYKRISX₁₀EGARDLISRLLKHNPSQRPMLREVLEHPWITANSSKPSNAQNK ESASKQS (EDP2, SEQ ID NO: 51); wherein X₁, X₂, X₃, X₄, X₅, X₆, X₇, X₈, X₉, and X₁₀ each comprise a sequence of at least 2 amino acids and at most 10 amino acids. Optionally, the protein can have amino acid sequence that is at least 80%, 85%, 90%, 95%, 96%, 97%, 98% or 99% identical to the amino acid sequence of EDP2. Further optionally the protein has the amino acid sequence of EDP2.

In some configurations, a protein having the EDP2 sequence (or homologous sequence) is an epitope display protein and one or more of X₁, X₂, X₃, X₄, X₅, X₆, X₇, X₈, X₉, and X₁₀ includes a target epitope. Any one of X₁, X₂, X₃, X₄, X₅, X₆, X₇, X₈, X₉, or X₁₀ can independently include a sequence of at least 2, 3, 4, 5, 6, 7, 8, 9 or 10 amino acids. Alternatively or additionally, any one of X₁, X₂, X₃, X₄, X₅, X₆, X₇, X₈, X₉, or X₁₀ of a protein having the EDP2 sequence, or homologue thereof, a can independently include a sequence of at most 10, 9, 8, 7, 6, 5, 4, 3 or 2 amino acids. Exemplary target epitopes that can be included in a protein having the EDP2 sequence (or homologous sequence), such as the proteins listed in Table 2, can include, but are not limited to, HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

A protein having the EDP2 sequence (or homologous sequence) can have an amino acid sequence that is substantially different from the amino acid sequence of Human Aurora Kinase A (see Table 2). For example, a protein having the EDP2 sequence (or homologous sequence) can have a sequence that is at most 90%, 85%, 80%, 75%, 70% or less identical to the amino acid sequence of Human Aurora Kinase A. Alternatively or additionally, the sequence can be at least 75%, 80%, 85%, 90%, 95%, 96%, 97%, or 98% identical to the amino acid sequence of Human Aurora Kinase A. Comparison of amino acid sequences of Human Aurora Kinase A and a protein having the EDP2 sequence (or homologous sequence) can span the full sequence of the Human Aurora Kinase A protein or can omit sequence regions corresponding to at least one, some or all of the loop regions in the secondary structure of the Human Aurora Kinase A protein. Optionally, a comparison of amino acid sequences for Human Aurora Kinase A and a protein having the EDP2 sequence (or homologous sequence) can omit sequence regions corresponding to at least one, some or all of X₁, X₂, X₃, X₄, X₅, X₆, X₇, X₈, X₉, or X₁₀ of the latter.

Optionally, a protein having the EDP2 sequence (or homologous sequence) includes at least one of the following structural features: X₁ is not GKF, X₂ is not KQSKF (SEQ ID NO: 65), X₃ is not LRHP (SEQ ID NO: 66), X₄ is not DAT, X₅ is not SKFD (SEQ ID NO: 67), X₆ is not GSAGE (SEQ ID NO: 68), X₇ is not PSSRRTTLCGT (SEQ ID NO: 69), X₈ is not EGRMHD (SEQ ID NO: 70), X₉ is not EANT (SEQ ID NO: 71), or X₁₀ is not RVEFTFPDFVT (SEQ ID NO: 72). For example, the protein can include at least 1, 2, 3, 4, 5, 6, 7, 8, 9 or 10 of the foregoing structural features. Alternatively or additionally, the protein can include at most 1, 2, 3, 4, 5, 6, 7, 8, 9 or 10 of the foregoing structural features. Accordingly, the protein can include 1, 2, 3, 4, 5, 6, 7, 8, 9 or 10 of the foregoing structural features.

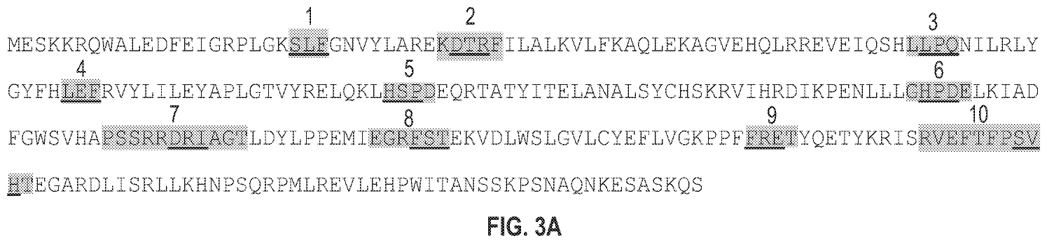

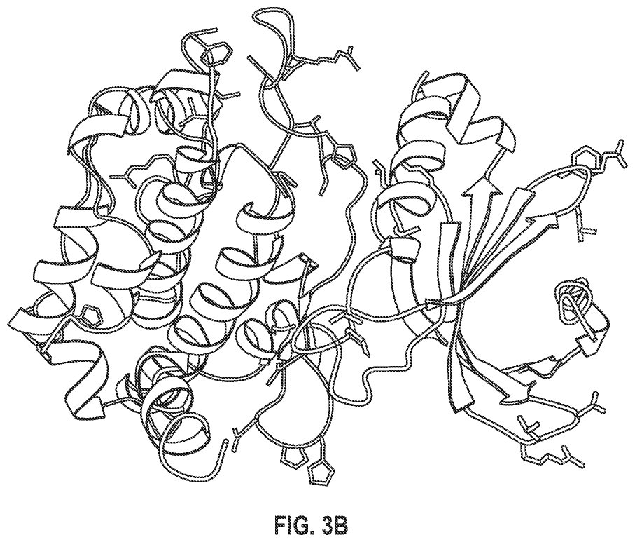

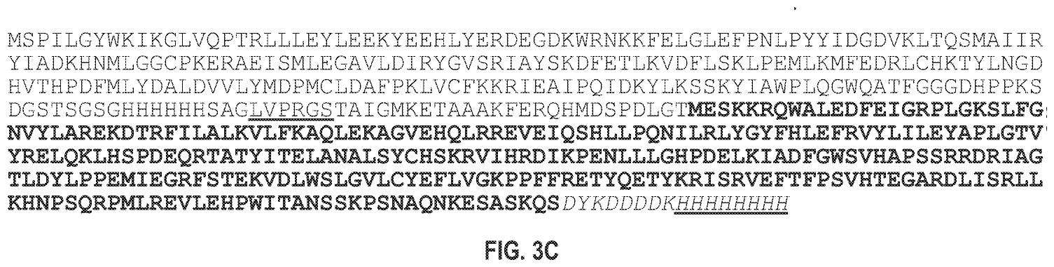

The EDP2-10 epitope display protein includes ten trimer epitopes displayed in 10 loop regions of the EDP2 epitope display structure motif. FIG. 3A shows the amino acid sequence for the EDP2-10 epitope display protein. The loop regions are highlighted in gray shading. The trimer epitopes are underlined and include SLF (X₁), DTR (X₂), LPQ (X₃), LEF (X₄), HSP (X₅), HPD (X₆), DRI (X₇), FST (X₈), FRE (X₉), and SVH (X₁₀). FIG. 3B shows the tertiary and secondary structure predicted for the EDP2-10 epitope display protein, wherein the side chains for amino acids of several epitopes are shown. An epitope display protein can include the EDP2 epitope display structure motif (i.e., the regions of regular secondary structure, an exemplary view of which is shown in FIG. 3B) and at least 1, 2, 3, 4, 5, 6, 7, 8, 9 or 10 of the epitopes of EDP2-10. Alternatively or additionally, an epitope display protein can include the EDP2 epitope display structure motif and at most 1, 2, 3, 4, 5, 6, 7, 8, 9 or 10 of the epitopes of EDP2-10. As a further option, an epitope display protein having the EDP2 epitope display structure motif can include a pre-sequence or post-sequence. The pre- or post-sequence can include, for example, a cysteine residue, an affinity tag or a protease cleavage site. The cysteine residue can be unique to the epitope display protein, for example, providing a known position for sulfur-based modification of the protein. The affinity tag can be glutathione-S-transferase or His-Tag, for example, as shown in FIG. 3C, or any other functional affinity tag such as those set forth herein. The protease cleavage site can be a thrombin site, for example, as shown in FIG. 3C, or any other functional protease cleavage site known in the art. As exemplified in FIG. 3C, a protease cleavage site can be positioned between the epitope display structure motif and one or both of the cysteine and affinity tag. As such protease cleavage can release one or both of the cysteine and affinity tag from the epitope display structure motif.

An epitope display protein can have an amino acid sequence that is at least 75% identical to EDP2-10. Optionally, the protein can have amino acid sequence that is at least 80%, 85%, 90%, 95%, 96%, 97%, 98% or 99% identical to the amino acid sequence of EDP2-10. Further optionally the protein has amino acid sequence of EDP2-10.

An epitope display protein can have an amino acid sequence that is at least 75% identical to MESKKRQWALEDFEIGRPLGKX₁GNVYLAREKQSKFILALKVLFKAQLEKAGVEHQLRR EVEIQSHLRHPNILRLYGYFHDATRVYLILEYAPLGTVYRELQKLSKFDEQRTATYITELA NALSYCHSKRVIHRDIKPENLLLGSAGELKIADFGWSVHAPSSRRTTLCGTLDYLPPEMI EGRMHDEKVDLWSLGVLCYEFLVGKPPFEANTYQETYKRISR VEFTFPDFVTEGARDLI SRLLKHNPSQRPMLREVLEHPWITANSSKPSNCQNKESASKQS (EDP2X₁, SEQ ID NO: 55); wherein X₁ includes at most 10, 9, 8, 7, 6, 5, 4, 3 or 2 amino acids. Alternatively or additionally, X₁ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₁. Further optionally the protein has amino acid sequence of EDP2X₁

An epitope display protein can have an amino acid sequence that is at least 75% identical to MESKKRQWALEDFEIGRPLGKGKFGNVYLAREX₂ILALKVLFKAQLEKAGVEHQLRRE VEIQSHLRHPNILRLYGYFHDATRVYLILEYAPLGTVYRELQKLSKFDEQRTATYITELA NALSYCHSKRVIHRDIKPENLLLGSAGELKIADFGWSVHAPSSRRTTLCGTLDYLPPEMI EGRMHDEKVDLWSLGVLCYEFL VGKPPFEANTYQETYKRISR VEFTFPDFVTEGARDLI SRLLKHNPSQRPMLREVLEHPWITANSSKPSNCQNKESASKQS (EDP2X₂, SEQ ID NO: 56); wherein X₂ includes at most 10, 9, 8, 7, 6, 5, 4, 3 or 2 amino acids. Alternatively or additionally, X₂ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₂. Further optionally the protein has amino acid sequence of EDP2X₂.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHX₃NILRLYGYFHDATRVYLILEYAPLGTVYRELQKLSKFDEQRTATYITELA NALSYCHSKRVIHRDIKPENLLLGSAGELKIADFGWSVHAPSSRRTTLCGTLDYLPPEMI EGRMHDEKVDLWSLGVLCYEFL VGKPPFEANTYQETYKRISR VEFTFPDFVTEGARDLI SRLLKHNPSQRPMLREVLEHPWITANSSKPSNCQNKESASKQS (EDP2X₃, SEQ ID NO: 57); wherein X₃ includes at most 10, 9, 8, 7, 6, 5, 4, 3 or 2 amino acids. Alternatively or additionally, X₃ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₃. Further optionally the protein has amino acid sequence of EDP2X₃.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHX₄RVYLILEYAPLGTVYRELQKLSKFDEQRTATYITEL ANALSYCHSKRVIHRDIKPENLLLGSAGELKIADFGWSVHAPSSRRTTLCGTLDYLPPEM IEGRMHDEKVDLWSLGVLCYEFL VGKPPFEANTYQETYKRISRVEFTFPDFVTEGARDLI SRLLKHNPSQRPMLREVLEHPWITANSSKPSNCQNKESASKQS (EDP2X+, SEQ ID NO: 58); wherein X₄ includes at most 10, 9, 8, 7, 6, 5, 4, 3 or 2 amino acids. Alternatively or additionally, X₄ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₄. Further optionally the protein has amino acid sequence of EDP2X₄.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLX₅EQRTATYITELA NALSYCHSKRVIHRDIKPENLLLGSAGELKIADFGWSVHAPSSRRTTLCGTLDYLPPEMI EGRMHDEKVDLWSLGVLCYEFLVGKPPFEANTYQETYKRISR VEFTFPDFVTEGARDLI SRLLKHNPSQRPMLREVLEHPWITANSSKPSNCQNKESASKQS (EDP2X₅, SEQ ID NO: 59); wherein X₅ includes at most 10, 9, 8, 7, 6, 5, 4, 3 or 2 amino acids. Alternatively or additionally, X₅ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₅. Further optionally the protein has amino acid sequence of EDP2X₅.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLSKFDEQRTATYITE LANALSYCHSKRVIHRDIKPENLLLX₆LKIADFGWSVHAPSSRRTTLCGTLDYLPPEMIEG RMHDEKVDLWSLGVLCYEFLVGKPPFEANTYQETYKRISRVEFTFPDFVTEGARDLISR LLKHNPSQRPMLREVLEHPWITANSSKPSNCQNKESASKQS (EDP2X₆, SEQ ID NO:60); wherein X₆ includes at most 10, 9, 8, 7, 6, 5, 4, 3 or 2 amino acids. Alternatively or additionally, X₆ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₆. Further optionally the protein has amino acid sequence of EDP2X₆.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLSKFDEQRTATYITE LANALSYCHSKRVIHRDIKPENLLLGSAGELKIADFGWSVHAX₇LDYLPPEMIEGRMHDE KVDLWSLGVLCYEFLVGKPPFEANTYQETYKRISRVEFTFPDFVTEGARDLISRLLKHNP SQRPMLREVLEHPWITANSSKPSNCQNKESASKQS (EDP2X₇, SEQ ID NO:61); wherein X₇ includes at most 10, 9, 8, 7, 6, 5, 4, 3 or 2 amino acids. Alternatively or additionally, X₇ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₇. Further optionally the protein has amino acid sequence of EDP2X₇.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLSKFDEQRTATYITE LANALSYCHSKRVIHRDIKPENLLLGSAGELKIADFGWSVHAPSSRRTTLCGTLDYLPPE MIX₈EKVDLWSLGVLCYEFLVGKPPFEANTYQETYKRISRVEFTFPDFVTEGARDLISRLL KHNPSQRPMLREVLEHPWITANSSKPSNCQNKESASKQS (EDP2X₈, SEQ ID NO:62); wherein X₈ includes at most 10, 9, 8, 7, 6, 5, 4, 3 or 2 amino acids. Alternatively or additionally, X₈ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₈. Further optionally the protein has amino acid sequence of EDP2X₈.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLSKFDEQRTATYITE LANALSYCHSKRVIHRDIKPENLLLGSAGELKIADFGWSVHAPSSRRTTLCGTLDYLPPE MIEGRMHDEKVDLWSLGVLCYEFLVGKPPFX₉YQETYKRISRVEFTFPDFVTEGARDLIS RLLKHNPSQRPMLREVLEHPWITANSSKPSNCQNKESASKQS (EDP2X₉, SEQ ID NO:63); wherein X₉ includes at most 10, 9, 8, 7, 6, 5, 4, 3 or 2 amino acids. Alternatively or additionally, X₉ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₉. Further optionally the protein has amino acid sequence of EDP2X₉.

An epitope display protein can have an amino acid sequence that is at least 75% identical to MESKKRQWALEDFEIGRPLGKGKFGNVYLAREKQSKFILALKVLFKAQLEKAGVEHQL RREVEIQSHLRHPNILRLYGYFHDATRVYLILEYAPLGTVYRELQKLSKFDEQRTATYITE LANALSYCHSKRVIHRDIKPENLLLGSAGELKIADFGWSVHAPSSRRTTLCGTLDYLPPE MIEGRMHDEKVDLWSLGVLCYEFLVGKPPFEANTYQETYKRISX₁₀EGARDLISRLLKHN PSQRPMLREVLEHPWITANSSKPSNCQNKESASKQS (EDP2X₁₀, SEQ ID NO:64); wherein X₁₀ includes at most 10, 9, 8, 7, 6, 5, 4, 3 or 2 amino acids. Alternatively or additionally, X₁₀ includes at least 2, 3, 4, 5, 6, 7, 8, 9, or 10 amino acids. Optionally, the protein can have amino acid sequence that is at least 80%, 85%, 90%, 95%, 96%, 97%, 98% or 99% identical to the amino acid sequence of EDP2X₁₀. Further optionally the protein has amino acid sequence of EDP2X₁₀.

Optionally, a protein having a sequence selected from EDP2, the sequences listed in Table 2, or a homologous sequence thereof, can include an epitope that is flanked with a glycine residue on the amino terminal and/or carboxy terminal side of the epitope. For example, a glycine can be present at a position in the amino acid sequence of an epitope display protein that occurs between a region of regular secondary structure and an epitope. For a protein having the sequence of EDP2, EDP2X₁, or homologue thereof, X₁ can include the amino acid sequence GX_{1D}, wherein X_{1D} includes a sequence of at least 2, 3, 4, 5 or 6 amino acids. Alternatively or additionally, X_{1D} can include a sequence of at most 6, 5, 4, 3 or 2 amino acids. Optionally, X₁ can include the amino acid sequence X_{1E}G, wherein X_{1E} includes a sequence of at least 2, 3, 4, 5 or 6 amino acids. Alternatively or additionally, X_{1E} can include a sequence of at most 6, 5, 4, 3 or 2 amino acids. As a further option, X₁ can include the amino acid sequence GX_{1F}G, wherein X_{1F} includes a sequence of at least 2, 3, 4, or 5 amino acids. Alternatively or additionally, X_{1F} can include a sequence of at most 5, 4, 3 or 2 amino acids.

For a protein having the sequence of EDP2, EDP2X₂, or homologue thereof, X₂ can include the amino acid sequence GX_{2D}, wherein X_{2D} includes a sequence of at least 2, 3, 4, 5 or 6 amino acids. Alternatively or additionally, X_{2D} can include a sequence of at most 6, 5, 4, 3 or 2 amino acids. Optionally, X₂ can include the amino acid sequence X_{2E}G, wherein X_{2E} includes a sequence of at least 2, 3, 4, 5 or 6 amino acids. Alternatively or additionally, X_{2E} can include a sequence of at most 6, 5, 4, 3 or 2 amino acids. As a further option, X₂ can include the amino acid sequence GX_{2F}G, wherein X_{2F} includes a sequence of at least 2, 3, 4, or 5 amino acids. Alternatively or additionally, X_{2F} can include a sequence of at most 5, 4, 3 or 2 amino acids.

For a protein having the sequence of EDP2, EDP2X₃, or homologue thereof, X₃ can include the amino acid sequence GX_{3D}, wherein X_{3D} includes a sequence of at least 2, 3, 4, 5 or 6 amino acids. Alternatively or additionally, X_{3D} can include a sequence of at most 6, 5, 4, 3 or 2 amino acids. Optionally, X₃ can include the amino acid sequence X_{3E}G, wherein X_{3E} includes a sequence of at least 2, 3, 4, 5 or 6 amino acids. Alternatively or additionally, X_{3E} can include a sequence of at most 6, 5, 4, 3 or 2 amino acids. As a further option, X₃ can include the amino acid sequence GX_{3F}G, wherein X_{3F} includes a sequence of at least 2, 3, 4, or 5 amino acids. Alternatively or additionally, X_{3F} can include a sequence of at most 5, 4, 3 or 2 amino acids.

For a protein having the sequence of EDP2, EDP2X₄, or homologue thereof, X₄ can include the amino acid sequence GX_{4D}, wherein X_{4D} includes a sequence of at least 2, 3, 4, 5 or 6 amino acids. Alternatively or additionally, X_{4D} can include a sequence of at most 6, 5, 4, 3 or 2 amino acids. Optionally, X₄ can include the amino acid sequence X_{4E}G, wherein X_{4E} includes a sequence of at least 2, 3, 4, 5 or 6 amino acids. Alternatively or additionally, X_{4E} can include a sequence of at most 6, 5, 4, 3 or 2 amino acids. As a further option, X₄ can include the amino acid sequence GX_{4F}G, wherein X_{4F} includes a sequence of at least 2, 3, 4, or 5 amino acids. Alternatively or additionally, X_{4F} can include a sequence of at most 5, 4, 3 or 2 amino acids.

For a protein having the sequence of EDP2, EDP2X₅, or homologue thereof, X₅ can include the amino acid sequence GX_{5D}, wherein X_{5D} includes a sequence of at least 2, 3, 4, 5 or 6 amino acids. Alternatively or additionally, X_{5D} can include a sequence of at most 6, 5, 4, 3 or 2 amino acids. Optionally, X₅ can include the amino acid sequence X_{5E}G, wherein X_{5E} includes a sequence of at least 2, 3, 4, 5 or 6 amino acids. Alternatively or additionally, X_{5E} can include a sequence of at most 6, 5, 4, 3 or 2 amino acids. As a further option, X₅ can include the amino acid sequence GX_{5F}G, wherein X_{5F} includes a sequence of at least 2, 3, 4, or 5 amino acids. Alternatively or additionally, X_{5F} can include a sequence of at most 5, 4, 3 or 2 amino acids.

For a protein having the sequence of EDP2, EDP2X₆, or homologue thereof, X₆ can include the amino acid sequence GX_{6D}, wherein X_{6D} includes a sequence of at least 2, 3, 4, 5 or 6 amino acids. Alternatively or additionally, X_{6D} can include a sequence of at most 6, 5, 4, 3 or 2 amino acids. Optionally, X₆ can include the amino acid sequence X_{6E}G, wherein X_{6E} includes a sequence of at least 2, 3, 4, 5 or 6 amino acids. Alternatively or additionally, X_{6E} can include a sequence of at most 6, 5, 4, 3 or 2 amino acids. As a further option, X₆ can include the amino acid sequence GX_{6F}G, wherein X_{6F} includes a sequence of at least 2, 3, 4, or 5 amino acids. Alternatively or additionally, X_{6F} can include a sequence of at most 5, 4, 3 or 2 amino acids.

For a protein having the sequence of EDP2, EDP2X₇, or homologue thereof, X₇ can include the amino acid sequence GX_{7D}, wherein X_{7D} includes a sequence of at least 2, 3, 4, 5 or 6 amino acids. Alternatively or additionally, X_{7D} can include a sequence of at most 6, 5, 4, 3 or 2 amino acids. Optionally, X₇ can include the amino acid sequence X_{7E}G, wherein X_{7E} includes a sequence of at least 2, 3, 4, 5 or 6 amino acids. Alternatively or additionally, X_{7E} can include a sequence of at most 6, 5, 4, 3 or 2 amino acids. As a further option, X₇ can include the amino acid sequence GX_{7F}G, wherein X_{7F} includes a sequence of at least 2, 3, 4, or 5 amino acids. Alternatively or additionally, X_{7F} can include a sequence of at most 5, 4, 3 or 2 amino acids.

For a protein having the sequence of EDP2, EDP2X₈, or homologue thereof, X₈ can include the amino acid sequence GX_{8D}, wherein X_{8D} includes a sequence of at least 2, 3, 4, 5 or 6 amino acids. Alternatively or additionally, X_{8D} can include a sequence of at most 6, 5, 4, 3 or 2 amino acids. Optionally, Xx can include the amino acid sequence X_{8E}G, wherein X_{8E} includes a sequence of at least 2, 3, 4, 5 or 6 amino acids. Alternatively or additionally, X_{8E} can include a sequence of at most 6, 5, 4, 3 or 2 amino acids. As a further option, Xx can include the amino acid sequence GX_{8F}G, wherein X_{8F} includes a sequence of at least 2, 3, 4, or 5 amino acids. Alternatively or additionally, X_{8F} can include a sequence of at most 5, 4, 3 or 2 amino acids.

For a protein having the sequence of EDP2, EDP2X₉, or homologue thereof, X₉ can include the amino acid sequence GX_{9D}, wherein X_{9D} includes a sequence of at least 2, 3, 4, 5 or 6 amino acids. Alternatively or additionally, X_{9D} can include a sequence of at most 6, 5, 4, 3 or 2 amino acids. Optionally, X₉ can include the amino acid sequence X_{9E}G, wherein X_{9E} includes a sequence of at least 2, 3, 4, 5 or 6 amino acids. Alternatively or additionally, X_{9E} can include a sequence of at most 6, 5, 4, 3 or 2 amino acids. As a further option, X₉ can include the amino acid sequence GX_{9F}G, wherein X_{9F} includes a sequence of at least 2, 3, 4, or 5 amino acids. Alternatively or additionally, X_{9F} can include a sequence of at most 5, 4, 3 or 2 amino acids.

For a protein having the sequence of EDP2, EDP2X₁₀, or homologue thereof, X₁₀ can include the amino acid sequence GX_{10D}, wherein X_{10D} includes a sequence of at least 2, 3, 4, 5 or 6 amino acids. Alternatively or additionally, X_{10D} can include a sequence of at most 6, 5, 4, 3 or 2 amino acids. Optionally, X₁₀ can include the amino acid sequence X_{10E}G, wherein X_{10E} includes a sequence of at least 2, 3, 4, 5 or 6 amino acids. Alternatively or additionally, X_{10E} can include a sequence of at most 6, 5, 4, 3 or 2 amino acids. As a further option, X₁₀ can include the amino acid sequence GX_{10F}G, wherein X_{10F} includes a sequence of at least 2, 3, 4, or 5 amino acids. Alternatively or additionally, X_{10F} can include a sequence of at most 5, 4, 3 or 2 amino acids.

A protein having the EDP2 sequence (or homologous sequence), can display a given epitope of interest in one or more of X₁, X₂, X₃, X₄, X₅, X₆, X₇, X₈, X₉, or X₁₀; two or more of X₁, X₂, X₃, X₄, X₅, X₆, X₇, X₈, X₉, or X₁₀; three or more of X₁, X₂, X₃, X₄, X₅, X₆, X₇, X₈, X₉, or X₁₀; four or more of X₁, X₂, X₃, X₄, X₅, X₆, X₇, X₈, X₉, or X₁₀; five or more of X₁, X₂, X₃, X₄, X₅. X₆, X₇, X₈, X₉, or X₁₀; six or more of X₁, X₂, X₃, X₄, X₅, X₆, X₇, X₈, X₉, or X₁₀; seven or more of X₁, X₂, X₃, X₄, X₅, X₆, X₇, X₈, X₉, or X₁₀; eight or more of X₁, X₂, X₃, X₄, X₅, X₆, X₇, X₈, X₉, or X₁₀; nine or more of X₁, X₂, X₃, X₄, X₅, X₆, X₇, X₈, X₉, or X₁₀; or ten or more of X₁, X₂, X₃, X₄, X₅, X₆, X₇, X₈, X₉, or X₁₀. Alternatively or additionally, a protein having the EDP2 sequence (or homologous sequence), can display a given epitope of interest in ten or fewer of X₁, X₂, X₃, X₄, X₅, X₆, X₇, X₈, X₉, or X₁₀; nine or fewer of X₁, X₂, X₃, X₄, X₅, X₆, X₇, X₈, X₉, or X₁₀; eight or fewer of X₁, X₂, X₃, X₄, X₅, X₆, X₇, X₈, X₉, or X₁₀; seven or fewer of X₁, X₂, X₃, X₄, X₅, X₆, X₇, X₈, X₉, or X₁₀; six or fewer of X₁, X₂, X₃, X₄, X₅, X₆, X₇, X₈, X₉, or X₁₀; five or fewer of X₁, X₂, X₃, X₄, X₅, X₆, X₇, X₈, X₉, or X₁₀; four or fewer of X₁, X₂, X₃, X₄, X₅, X₆, X₇, X₈, X₉, or X₁₀; three or fewer of X₁, X₂, X₃, X₄, X₅, X₆, X₇, X₈, X₉, or X₁₀; two or fewer of X₁, X₂, X₃, X₄, X₅. X₆, X₇, X₈, X₉, or X₁₀; or no more than one of X₁, X₂, X₃, X₄, X₅, X₆, X₇, X₈, X₉, or X₁₀. As such, the same epitope can be present in multiple loop regions of an epitope display protein.

Optionally, a protein having the EDP2 sequence (or homologous sequence) can display different epitopes of interest in two or more of X₁, X₂, X₃, X₄, X₅, X₆, X₇, X₈, X₉, or X₁₀; three or more of X₁, X₂, X₃, X₄, X₅, X₆, X₇, X₈, X₉, or X₁₀; four or more of X₁, X₂, X₃, X₄, X₅, X₆, X₇, X₈, X₉, or X₁₀; five or more of X₁, X₂, X₃, X₄, X₅, X₆, X₇, X₈, X₉, or X₁₀; six or more of X₁, X₂, X₃, X₄, X₅, X₆, X₇, X₈, X₉, or X₁₀; seven or more of X₁, X₂, X₃, X₄, X₅, X₆, X₇, X₈, X₉, or X₁₀; eight or more of X₁, X₂, X₃, X₄, X₅, X₆, X₇, X₈, X₉, or X₁₀; nine or more of X₁, X₂, X₃, X₄, X₅, X₆, X₇, X₈, X₉, or X₁₀; or ten or more of X₁, X₂, X₃, X₄, X₅, X₆, X₇, X₈, X₉, or X₁₀. Alternatively or additionally, a protein having the EDP2 sequence (or homologous sequence), can display different epitopes of interest in ten or fewer of X₁, X₂, X₃, X₄, X₅, X₆, X₇, X₈, X₉, or X₁₀; nine or fewer of X₁, X₂, X₃, X₄, X₅, X₆, X₇, X₈, X₉, or X₁₀; eight or fewer of X₁, X₂, X₃, X₄, X₅, X₆, X₇, X₈, X₉, or X₁₀; seven or fewer of X₁, X₂, X₃, X₄, X₅, X₆, X₇, X₈, X₉, or X₁₀; six or fewer of X₁, X₂, X₃, X₄, X₅, X₆, X₇, X₈, X₉, or X₁₀; five or fewer of X₁, X₂, X₃, X₄, X₅, X₆, X₇, X₈, X₉, or X₁₀; four or fewer of X₁, X₂, X₃, X₄, X₅, X₆, X₇, X₈, X₉, or X₁₀; three or fewer of X₁, X₂, X₃, X₄, X₅. X₆, X₇, X₈, X₉, or X₁₀; or two of X₁, X₂, X₃, X₄, X₅, X₆, X₇, X₈, X₉, or X₁₀.

Amino acids that are present in an epitope display protein are typically L-amino acids. For example, epitopes in proteins set forth herein can be L-amino acids. However, D-amino acids can be used in an epitope display protein, for example, in the epitopes therein. Epitope display proteins will typically include amino acids selected from among the standard 20 amino acids encoded by the human genome or other genome of interest. For example, an epitope of an epitope display protein can include amino acids encoded by the human genome. Optionally, the amino acids that are included in an epitope display protein (e.g. in an epitope thereof) can include essential amino acids.

Optionally, one or more amino acids included in an epitope display protein, for example, in an epitope thereof, can include a post-translational modification (PTM) moiety. The PTM moiety can be added by a biological system, by one or more components of a biological system or by a synthetic procedure. In some configurations, an epitope display protein can include an epitope that is modifiable to generate a post-translational modification. A PTM moiety may be present in the epitope or absent from the epitope to suit a desired use of the epitope display protein. An epitope can include an amino acid of a type that is prone to post-translational modification and in some cases can include a sequence of amino acids that is recognized by, or otherwise facilitates, modification by an enzyme or other biochemical agent. Exemplary PTM moieties include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization.

A post-translational modification may occur at a particular type of amino acid residue. Optionally, the amino acid residue can be located in an epitope of an epitope display protein. For example, a phosphoryl moiety can be present on a serine, threonine, tyrosine, histidine, cysteine, lysine, aspartate or glutamate residue. In another example, an acetyl moiety can be present on the N-terminus or on a lysine of a protein. In another example, a serine or threonine residue of a protein can have an O-linked glycosyl moiety, or an asparagine residue of a protein can have an N-linked glycosyl moiety. In another example, a proline, lysine, asparagine, aspartate or histidine amino acid of a protein can be hydroxylated. In another example, a protein can be methylated at an arginine or lysine amino acid. In another example, a protein can be ubiquitinated at the N-terminal methionine or at a lysine amino acid. It will be understood that an epitope of the present disclosure can be devoid of one or more of the PTM moieties set forth herein. A method of the present disclosure can include a step of modifying one or more epitopes, for example, by adding a PTM moiety or removing a PTM moiety.

An epitope display protein of the present disclosure can be devoid of cysteine residues. For example, the GHSPG₅, GDPYGs and GWNKs proteins are devoid of cysteine residues. The absence of cysteine residues can be useful, for example, to avoid unwanted crosslinking of epitope display proteins to each other or to other proteins having cysteine residues. This can be particularly useful in oxidizing environments. The absence of cysteines can also render an epitope display protein inert to chemistries that target sulfurs, such as chemistries used to modify other proteins via reaction with cysteines. In some configurations, the regular secondary structure regions of an epitope display protein can be devoid of cysteines. In other words, the epitope display structure motif of an epitope display protein can be devoid of cysteines. Examples of epitope display proteins having epitope display structure motifs that lack a cysteine include EDP1, EDP1X₁, EDP1X₂, EDP1X₃, EDP1X₄, and EDP1X₅.

Alternatively, an epitope display protein of the present disclosure can include one or more cysteine residues. The presence of one or more cysteine residues can facilitate modifications that target cysteine, such as addition of a label, or attachment to a particle, solid support, or other protein. In some configurations, an epitope display protein can include a single cysteine (i.e. one and only one cysteine). This can provide a pre-selected location for spatially targeted modification of the epitope display protein. For example, a cysteine can be present at a location in the tertiary structure of an epitope display protein that is adequately distant from an epitope to avoid interfering with interaction of the epitope with an affinity reagent. More specifically, the cysteine can be linked to a moiety (e.g. a label, particle, solid support, or other protein) via a linker that is positioned to avoid interfering with binding of an affinity reagent to an epitope. Optionally, an epitope display protein can include a cysteine at or near the amino terminus or carboxy terminus. Examples of epitope display proteins having a cysteine residue in a terminal region include those having the pre-sequence MCGHHHHHHGWSENLYFQ (SEQ ID NO: 73) in Table 1. In some cases, an epitope display protein, or epitope display structure motif (e.g. regions of regular secondary structure) thereof, can include at least 1, 2, 3 or more cysteines. Alternatively or additionally, an epitope display protein, or epitope display structure motif (e.g. regions of regular secondary structure) thereof, can include at most 3, 2, or 1 cysteine(s).

An epitope display protein can include an affinity tag. An affinity tag can bind to a receptor or ligand to facilitate purification or detection of the epitope display protein. An affinity tag can be located at or near a terminus (e.g. amino terminus or carboxy terminus) of the epitope display protein. For example, an affinity tag of an epitope display protein can be located, in the primary structure of the protein, between the amino terminus and the epitope display structure motif or between the carboxy terminus and the epitope display structure motif. Examples of epitope display proteins having affinity tags include those having the pre-sequence

MCGHHHHHHGWSENLYFQ in Table 1 (here the affinity tag is the polyhistidine motif which has affinity for divalent metal cations such as Mn²⁺, Fe²⁺, Co²⁺, Ni²⁺, and Cu²⁺) and those having the pre-sequence MSPILGYWKIKGLVQPTRLLLEYLEEKYEEHLYERDEGDKWRNKKFELGLEFPNLPYYI DGDVKLTQSMAIIRYIADKHNMLGGCPKERAEISMLEGAVLDIRYGVSRIAYSKDFETLK VDFLSKLPEMLKMFEDRLCHKTYLNGDHVTHPDFMLYDALDVVLYMDPMCLDAFPKL VCFKKRIEAIPQIDKYLKSSKYIAWPLQGWQATFGGGDHPPKSDGSTSGSGHHHHHHSA GLVPRGSTAIGMKETAAAKFERQHMDSPDLGT (SEQ ID NO: 74; here the affinity tag is glutathione-S-transferase which has affinity for glutathione). Other useful affinity tags include, for example, a SpyTag™ which has affinity for SpyCatcher™ or, conversely, the SpyCatcher™ which has affinity for SpyTag™ (Zakeri et al., Proc Natl Acad Sci USA 109: E690-E697 (2012), which is incorporated herein by reference); a peptide, such as the FlagTag™ (Hopp et al., Bio/Technology 6:1204-1210 (1988), which is incorporated herein by reference) or Myc-Tag™ (Evan et al., Molecular and Cellular Biology. 5:3610-6 (1985), which is incorporated herein by reference), having affinity for an antibody; a peptide, such as StrepTag™ (Schmidt and Skerra Nature Protocols. 2:1528-35 (2007), which is incorporated herein by reference), having affinity for streptavidin, avidin or analogue thereof; or maltose binding protein having affinity for maltose (di Guan et al., Gene. 67:21-30 (1988), which is incorporated herein by reference). A fluorescent protein (e.g. green fluorescent protein (GFP), wavelength shifted mutant of GFP, or phycobiliprotein) can be similarly fused to an epitope display protein using well known molecular biology techniques.

An epitope display protein can include a protease recognition site. A protease recognition site of an epitope display protein can be located, in the primary structure of the protein, between the amino terminus and the epitope display structure motif or between the carboxy terminus and the epitope display structure motif. The epitope display protein can be treated with a protease that recognizes the site and cleaves the protein to separate the epitope display structure motif from the amino terminus or carboxy terminus, respectively. The protease recognition site can be positioned to allow separation of an epitope display protein motif, or epitope display structure motif thereof, from other functional regions such as a region having a cysteine residue, affinity tag, label, attachment to a non-proteinaceous material or the like. Exemplary proteins having a protease recognition site include those having the pre-sequence MCGHHHHHHGWSENLYFQ in Table 1 (here the protease recognition site is ENLYFQG, which is recognized by the TEV protease and cleaved between the Q and G residues) or those having the pre-sequence MSPILGYWKIKGLVQPTRLLLEYLEEKYEEHLYERDEGDKWRNKKFELGLEFPNLPYYI DGDVKLTQSMAIIRYIADKHNMLGGCPKERAEISMLEGAVLDIRYGVSRIAYSKDFETLK VDFLSKLPEMLKMFEDRLCHKTYLNGDHVTHPDFMLYDALDVVLYMDPMCLDAFPKL VCFKKRIEAIPQIDKYLKSSKYIAWPLQGWQATFGGGDHPPKSDGSTSGSGHHHHHHSA GLVPRGSTAIGMKETAAAKFERQHMDSPDLGT in Table 2 (SEQ ID NO: 74, here the protease recognition site is LVPRGS (SEQ ID NO: 75), which is recognized by thrombin and cleaved between the R and G residues).

An epitope display protein, or epitope display structure motif thereof, can be configured to have a predetermined number of lysine (K) residues. Moreover, lysines can be present at preselected locations in an epitope display protein, or epitope display structure motif thereof. Lysines have relatively reactive amino moieties in their side chains and are, thus, useful for attachment to labels, particle, solid supports or other substances. Engineering the number and/or position of lysine residues can provide the benefit of spatially controlled modification of the protein. For example, a lysine can be positioned at a location of an epitope display protein that is adequately separated from an epitope of interest to prevent modification of the lysine from interfering with binding of an affinity reagent to the epitope. An epitope display protein can be configured to lack lysines in all loop regions or in all loop regions that include an epitope of interest. Optionally, an epitope display protein, or epitope display structure motif thereof, can be configured to have no lysines or to have a single lysine (i.e. one and only one lysine). In some configurations, an epitope display protein, or epitope display structure motif thereof, can have at least 1, 2, 3, 4, 5, 6, 7, 8, 9, 10 or more lysine residues. Alternatively or additionally, an epitope display protein, or epitope display structure motif thereof, can have at most 10, 9, 8, 7, 6, 5, 4, 3, 2 or 1 lysine residues. The epitope display structure motif of the EDP1 protein includes seven lysine residues. The EDP1 protein, or epitope display structure motif thereof, can be engineered to include at most 7, 6, 5, 4, 3, 2 or 1 lysine residue. Alternatively or additionally, the EDP1 protein, or epitope display structure motif thereof, can be engineered to include at least 1, 2, 3, 4, 5, 6, 7 or more lysine residues. Lysine residues can be replaced by any of a variety of the 20 amino acids. A particularly useful replacement for lysine is arginine due to its similar size and charge. Optionally, all but one of the lysine residues of EDP1 can be replaced by an arginine or other residue. For example, all lysine residues of EDP1 except lysine 7, 10, 23, 34, 35, 42, or 43 can be replaced by an arginine or other amino acid residue. Indeed, any number and combination of lysines 7, 10, 23, 34, 35, 42, or 43 in EDP1 can be replaced by an arginine or other amino acid residue.

An epitope display protein of the present disclosure can be bound to an affinity reagent. The binding can occur between the affinity reagent and an epitope that is present in a loop region of the epitope display protein. For example, binding can occur between an affinity reagent and EDP1. The affinity reagent can be bound to an epitope present in X₁, X₂, X₃, X₄ or X₅ of the EDP1 sequence or a homologous sequence thereof. Any of a variety of affinity reagents can be bound to an epitope display protein including, but not limited to, an antibody, such as a full length antibody or functional fragment thereof (e.g., Fab′ fragment, F(ab′)₂ fragment, single-chain variable fragment (scFv), di-scFv, tri-scFv, or microantibody), aptamer (e.g. nucleic acid aptamer), affibody, affilin, affimer, affitin, alphabody, anticalin, avimer, miniprotein, DARPin, monobody, nanoCLAMP, lectin, or functional fragments thereof.

A complex containing an epitope display protein and affinity reagent can further include a label. For example, an affinity reagent that participates in a complex or that is otherwise used for binding to an epitope display protein can include a label. A label can be endogenous to the affinity reagent or other molecule to which it is attached. Alternatively, a label can be exogenous to an affinity reagent or other molecule to which it is attached, for example, being an artificial moiety or a moiety added using a synthetic process. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). In some cases, a label can be attached to an epitope display protein set forth herein. For example, a labeled epitope display protein can be used to detect the presence of an affinity reagent that recognizes an epitope present in the epitope display protein. Exemplary labels that can be attached to an affinity reagent or epitope display protein include, without limitation, a luminophore (e.g. fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A labeled complex that includes an affinity reagent and epitope display protein can be detected by virtue of signals produced by the label.

A complex between an affinity reagent and epitope display protein can be in fluid-phase. Alternatively, a complex between an affinity reagent and epitope display protein can be immobilized. For example, the epitope display protein can be immobilized on a solid support via covalent bonding or another attachment mechanism set forth herein, and the affinity reagent can be immobilized via binding to the epitope display protein. Thus, an affinity reagent can be attached to a solid support via binding to an epitope display protein on the solid support. The opposite configuration can also occur, wherein an affinity reagent is immobilized on a solid support via covalent bonding or another attachment mechanism set forth herein, and an epitope display protein is immobilized via binding to the affinity reagent. Thus, an epitope display protein can be attached to a solid support via binding to an affinity reagent on the solid support. An immobilized complex can be detected via a label that is present on any member of the complex, such as an epitope display protein or affinity reagent.

Optionally, an epitope display protein, affinity reagent or complex between an epitope display protein and affinity reagent can be attached to a particle. The particle can be a solid support particle, for example, including a material set forth herein in the context of solid supports. A particularly useful particle is a structured nucleic acid particle. A structured nucleic acid particle is a single- or multi-chain polynucleotide molecule having a compacted three-dimensional structure. The compacted three-dimensional structure can optionally be characterized in terms of hydrodynamic radius or Stoke's radius of the structured nucleic acid particle relative to a random coil or other non-structured state for a nucleic acid having the same sequence length as the structured nucleic acid particle. The compacted three-dimensional structure can optionally be characterized with regard to tertiary or quaternary structure. For example, a structured nucleic acid particle can be configured to have an increased number of interactions between polynucleotide strands or less distance between the strands, as compared to a nucleic acid molecule of similar length in a random coil or other non-structured state. In some configurations, the secondary structure of a structured nucleic acid particle can be configured to be denser than a nucleic acid molecule of similar length in a random coil or other non-structured state. A structured nucleic acid particle may contain DNA, RNA, PNA, modified or non-natural nucleic acids, or combinations thereof. A structured nucleic acid particle may include a plurality of oligonucleotides that hybridize to form the structured nucleic acid particle structure. The plurality of oligonucleotides in a structured nucleic acid particle may include oligonucleotides that are attached to other molecules (e.g., probes, analytes such as polypeptides, reactive moieties, or detectable labels) or are configured to be attached to other molecules (e.g., by functional groups). Exemplary structured nucleic acid particles include nucleic acid origami and nucleic acid nanoballs. Examples of useful structured nucleic acid particles and methods for their manufacture and use are set forth in U.S. Pat. Nos. 11,203,612 or 11,505,796 or US Pat. App. Pub. No. 2022/0162684 A1, each of which is incorporated herein by reference.

Nucleic acid origami is a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid. Examples of useful nucleic acid origami particles and methods for their manufacture and use are set forth in U.S. Pat. Nos. 11,203,612 or 11,505,796 or U.S. Pat. App. Pub. No. 2022/0162684 A1, each of which is incorporated herein by reference.

An epitope display protein, affinity reagent or complex between an epitope display protein and affinity reagent can be attached to an array. In some cases, an array can include a plurality of addresses. Individual addresses of an array can each be attached to an epitope display protein, affinity reagent or complex between an epitope display protein and affinity reagent. Individual addresses of an array can each be attached to a single molecule (e.g. a single epitope display protein or single affinity reagent) or to a single complex between an epitope display protein and affinity reagent. Thus, the single molecules can be individually resolved in an array. Alternatively, individual addresses of an array can each be attached to a plurality of epitope display proteins, a plurality of affinity reagents, or a plurality of complexes between epitope display proteins and affinity reagents. In some cases, the plurality of molecules at an address is an ensemble including multiple copies of the same molecule or complex. Alternatively, a plurality of different molecules or complexes can be present at an address of an array.

An array can include a plurality of different epitope display proteins. For example, the addresses of an array can be attached to different epitope display proteins, respectively. The different epitope display proteins can differ with respect to the epitopes present in the protein. For example, an array can include addresses that are attached to respective species of EDP1 proteins (e.g. a first address is attached to a species of EDP1 having a first epitope and a second address is attached to a species of EDP1 having a second epitope, wherein the first epitope is different from the second epitope). In some configurations, epitope display proteins in an array can differ with respect to the epitope display structure motif. For example, an array can include a first address that is attached to a species of EDP1 and a second address that is attached to a species of EDP2. An array can include one or more addresses attached to epitope display proteins, and the array can further include one or more addresses attached to proteins obtained from a biological sample. For example, the array can be attached to proteins from the proteome of an organism set forth herein.

It will be understood that a plurality of epitope display proteins, such as those having components or characteristics set forth above, need not be attached to an array. For example, a similar plurality of epitope display proteins can be present in a vessel, such as a test tube, well (e.g. in a multiwell plate), flow cell, microfluidic device, etc.; in a kit; in an apparatus; or attached to a particle or solid support.

One or more epitope display proteins can be provided in combination with one or more proteins from a proteome. The proteins can be attached to an array as set forth above but need not be. For example, the proteins can be mixed with one or more epitope display proteins in a fluid. The mixture can be present in vessel, kit or apparatus. A plurality of epitope display proteins can include at least 2, 3, 4, 5, 10, 15, 20, 25, 50, 100 different sequences, each sequence having the same epitope display structure motif and each sequence differing from the sequence of the other proteins of the plurality at one or more loop regions. For example, a plurality of epitope display proteins can include at least 2, 3, 4, 5, 10, 15, 20, 25, 50, 100 different sequences, each epitope display protein including the EDP1 sequence (or a homologous sequence) and each sequence differing from the sequence of the other proteins of the plurality at one or more of X₁, X₂, X₃, X₄ and X₅. In another example, a plurality of epitope display proteins can include at least 2, 3, 4, 5, 10, 15, 20, 25, 50, 100 different sequences, each epitope display protein including the EDP2 sequence (or a homologous sequence) and each sequence differing from the sequence of the other proteins of the plurality at one or more of X₁, X₂, X₃, X₄, X₅, X₆, X₇, X₈, X₉, or X₁₀.

Proteins that are used in a composition or method set forth herein can be obtained from any of a variety of organisms. Exemplary organisms from which a set of test polypeptides can be obtained include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A polypeptide can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid.

A plurality of proteins (e.g. from a proteome) can include at least 1, 10, 100, 1×10⁶, 1×10⁹, 1 mole (6.02214076× 10²³ molecules), or more protein molecules. Alternatively or additionally, a plurality of proteins may contain at most 1 mole, 1×10⁹, 1×10⁶, 1×10⁴, 100, 10 or, 1 protein molecules. A plurality of proteins can include a variety of different amino acid sequences. For example, the variety of full-length amino acid sequences in a plurality of test proteins can include substantially all different native-length amino acid sequences from a given organism or a subfraction thereof. A proteome or subfraction can have a complexity of at least 2, 5, 10, 100, 1×10³, 1×10⁴, 2×10⁴, 3×10⁴ or more different native-length amino acid sequences. Alternatively or additionally, a proteome, or subfraction thereof, can have a complexity that is at most 3×10⁴, 2×10⁴, 1×10⁴, 1×10³, 100, 10, 5, 2 or fewer different native-length amino acid sequences.

The diversity of a plurality of proteins (e.g. from a proteome) can include at least one representative for substantially all proteins encoded by the genome of the organism from which the sample was obtained, or a fraction thereof. For example, a plurality of proteins may contain at least one representative for at least 60%, 75%, 90%, 95%, 99%, or more of the proteins encoded by a particular organism. Alternatively or additionally, a plurality of proteins may contain a representative for at most 99%, 95%, 90%, 75%, 60% or fewer of the proteins encoded by a particular organism.

An epitope display protein can be used to evaluate and characterize affinity reagents. An epitope display protein can include epitopes for one or more affinity reagents of interest. A set of epitope display proteins can be configured to include multiple different proteins and each of the different proteins can contain multiple different epitopes. Moreover, one or more different epitopes can be redundantly present across multiple different epitope display proteins. For example, a particular epitope can be present in some or all different members of a set of epitope display proteins.

An epitope display protein or set of epitope display proteins can be used in any of a variety of contexts. A particularly useful context is a protein binding assay, wherein one or more epitope display proteins can be used to evaluate activity of one or more affinity reagents used in the assay. For example, an epitope display protein can serve as a positive or negative control for one or more affinity reagents used in an assay. A set of epitope display proteins can provide a plurality of positive and/or negative controls when determining binding strength or binding specificity of a set of affinity reagents. Similarly, an epitope display protein can serve as a quantitation standard for quantifying one or more proteins detected in an assay. For example, one or more epitope display proteins can be provided in known amounts to an assay for test proteins, the epitope display proteins and test proteins can be quantified, and the quantity of test proteins detected can be determined relative to the known amount of epitope display protein(s) provided to the assay. In some cases, one or more epitope display proteins can be provided in a series of different amounts and a standard curve can be generated from observed binding of affinity reagents to the series. The standard curve can be used to quantify test proteins detected using the affinity reagents.

Another context in which epitope display proteins of the present disclosure can be useful is preparation of affinity reagents. For example, an epitope display protein can serve as a target or bait for capturing an affinity reagent of interest in a selection or screening process. Alternatively, one or more epitope display proteins can be used in a negative selection step to remove or avoid affinity reagents having unwanted affinity for one or more epitopes. In another example, a fluid that contains an affinity reagent can be contacted with an immobilized epitope display protein, and an affinity reagent that binds the immobilized epitope display protein can be separated from the fluid. Separation can occur, for example, via affinity chromatography or solid-phase extraction. Similarly, an affinity reagent can be bound to a labeled epitope display protein to form a labeled complex and the label can be detected to monitor partitioning of the complex in one or more steps of a separation process.

In yet another context, one or more epitope display proteins can be used to characterize or assess quality of one or more affinity reagents. For example, binding of an affinity reagent to one or more epitope display proteins can be evaluated to determine epitope-binding specificity of the affinity reagent, probability of an affinity reagent binding particular epitope(s), strength of affinity reagent binding to particular epitope(s) (e.g. equilibrium dissociation constant or equilibrium association constant), kinetics of affinity reagent binding to particular epitope(s) (e.g. association rate, dissociation rate, kₒₙ or k_{off}). In some cases, specificity of an affinity reagent can be determined based on observed binding (or non-binding) to a set of epitope display proteins having a plurality of different epitopes.

The present disclosure provides a method of binding an affinity reagent to an epitope in an epitope display protein. In some configurations of the method, the epitope is present in a region of the primary structure of the epitope display protein that forms a loop in the secondary structure of the protein. As a further option, the epitope can be present in a region of the primary structure of the epitope display protein that forms a solvent-exposed loop in the tertiary or quaternary structure of the protein.

Optionally, a method of the present disclosure can be configured to include a step of binding an affinity reagent to a protein having an amino acid sequence that is at least 80% identical to EDP1, wherein X₁, X₂, X₃, X₄ and X₅ each comprise a sequence of at least 2 amino acids and at most 10 amino acids, and wherein the affinity reagent binds to the protein via X₁, X₂, X₃, X₄ or X₅.

Optionally, a method of the present disclosure can be configured to include a step of binding an affinity reagent to a protein having an amino acid sequence that is at least 80% identical to EDP2, wherein X₁, X₂, X₃, X₄, X₅, X₆, X₇, X₈, X₉, and X₁₀ each comprise a sequence of at least 2 amino acids and at most 10 amino acids, and wherein the affinity reagent binds to the protein via X₁, X₂, X₃, X₄, X₅, X₆, X₇, X₈, X₉, or X₁₀.

An affinity reagent, epitope display protein or complex between an affinity reagent and epitope display protein can include a label and the label can be detected in a method set forth herein using a detector that is appropriate for the signal produced by the label. For example, an optical detector can be used to detect luminescent labels or other labels that produce optical signals.

An affinity reagent or epitope display protein can be attached to a particle and/or solid support during one or more steps of a method set forth herein. For example, an affinity reagent or epitope display protein can be attached to a particle and/or solid support during a step of binding to an affinity reagent, during a detection step or during both steps. In some cases, an epitope display protein can be attached to a particle and/or solid support via an affinity reagent. For example, an affinity reagent can be attached to the particle and/or solid support and the epitope display protein can be bound to the attached affinity reagent. In other cases, an affinity reagent can be attached to a particle and/or solid support via an epitope display protein. For example, an epitope display protein can be attached to the particle and/or solid support and the affinity reagent can be bound to the attached affinity reagent. A complex between a solid support (and/or particle), affinity reagent and epitope display protein can be produced by (1) forming a binary complex between the affinity reagent and epitope display protein and then attaching the binary complex to the solid support (and/or particle); (2) attaching the affinity reagent to the solid support (and/or particle) and then binding the epitope display protein to the attached affinity reagent, or (3) attaching the epitope display protein to the solid support (and/or particle) and then binding the affinity reagent to the attached epitope display protein.

Optionally, an affinity reagent or epitope display protein can be attached to an address of an array. Detection can be carried out to distinguish individual addresses of the array. As such, an array can be used for multiplex detection of a plurality of affinity reagents and/or epitope display proteins. In some cases, individual addresses are each attached to a single affinity reagent or to a single epitope display protein. Accordingly, resolution of the addresses from each other during a detection step can function to resolve each affinity reagent from all other affinity reagents in the array or to resolve each epitope display protein from all other epitope display proteins in the array. An array can include a plurality of proteins, for example, a plurality of different proteins from a biological sample. The proteins from the sample can be attached to respective addresses of the array. Thus, resolution of the addresses from each other can resolve the sample proteins from each other and from epitope display proteins on the array.

An affinity reagent that is used in a method set forth herein can recognize an epitope that is present in an epitope display protein and also present in at least one protein from a sample. For example, the affinity reagent can bind to the epitope in the epitope display protein and in the sample protein(s). This can be due to the different proteins having the same epitope. Alternatively, the affinity reagent can be promiscuous, recognizing or binding to different epitopes. For example, the affinity reagent can recognize and bind to a first epitope that is present in an epitope display protein and a second epitope that is present in another protein. The second epitope can be biosimilar to the first epitope (e.g. the epitopes can be biosimilar according to the BLOSUM62 scoring matrix).

Optionally, a method set forth herein can further include a step of identifying a protein from a sample based on binding of an affinity reagent to the protein and to an epitope display protein. For example, the affinity reagent can have known recognition properties for a given epitope, the epitope binding protein can have the known epitope and the presence of the epitope in the sample protein can be determined from observation that the sample protein and the epitope binding protein both bind to the affinity reagent.

Epitope display proteins can be detected in a protein assay. Many protein assays, such as enzyme linked immunosorbent assay (ELISA), achieve high-confidence characterization of one or more proteins in a sample by exploiting high specificity binding of affinity reagents to the protein(s) and detecting the binding event while ignoring all other proteins in the sample. Binding assays can be carried out by detecting immobilized affinity reagents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection assays employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference. An epitope display protein set forth herein can be used in such assay formats.

A plurality of proteins can be assayed for binding to affinity reagents, for example, on single-molecule resolved protein arrays. Epitope display proteins can be included in the assay, for example, being attached to addresses in an array of sample proteins. Proteins (e.g. epitope display protein or sample protein) can be in a denatured state or native state when manipulated or detected in a method set forth herein. Exemplary assay formats that can be performed at a variety of plexity scales up to and including proteome scale are set forth in U.S. Pat. No. 10,473,654 or U.S. Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference. An epitope display protein set forth herein can be used in such assay formats.

Turning to the example of an array-based configuration, the identity of the sample protein at any given address is typically not known prior to performing the assay. The location and identity of one or more epitope display proteins may be known or unknown prior to performing the assay. The assay can be used to identify proteins (e.g. an epitope display protein or test protein) at one or more addresses in the array. A plurality of affinity reagents, optionally labeled (e.g. with fluorophores), can be contacted with the array, and the binding of affinity reagents can be detected at individual addresses to determine binding outcomes. A plurality of different affinity reagents can be delivered to the array and detected serially, such that each cycle detects binding outcomes for an individual affinity reagent. In some configurations, a plurality of affinity reagents can be detected in parallel, for example, when different affinity reagents are distinguishably labeled. The result of detecting binding of a plurality of affinity reagents to an array is a series of binding outcomes for each address of the array. Accordingly, the protein at each address will have a binding outcome profile that includes the series of binding outcomes. The binding profile can be decoded to identify the protein at each address.

In particular configurations, the methods can be used to identify a number of different proteins that exceeds the number of affinity reagents used. For example, the number of proteins identified can be at least 5×, 10×, 25×, 50×, 100× or more than the number of affinity reagents used. This can be achieved, for example, by (1) using promiscuous affinity reagents that bind to multiple different proteins suspected of being present in a given sample, and (2) subjecting the protein sample to a set of promiscuous affinity reagents that, taken as a whole, are expected to bind each protein in a different combination, such that each protein is expected to generate a unique binding profile. The binding profile can include positive binding outcomes (i.e. observation of binding between affinity reagent and protein). Optionally, the binding profile can also include negative binding outcomes (i.e. observation that a given affinity reagent did not bind to a given protein). Promiscuity of an affinity reagent can arise due to the affinity reagent recognizing an epitope that is known to be present in a plurality of different proteins. For example, epitopes having relatively short amino acid lengths such as dimers, trimers, tetramers or pentamers can be expected to occur in a substantial number of different proteins in a typical proteome. Alternatively or additionally, a promiscuous affinity reagent may recognize different epitopes (e.g. epitopes differing from each other with regard to amino acid composition or sequence). For example, a promiscuous affinity reagent that is designed or selected for its affinity toward a first trimer epitope may bind to a second epitope that has a different sequence of amino acids compared to the first epitope.

Although performing a single binding reaction between a promiscuous affinity reagent and a complex protein sample may yield ambiguous results regarding the identity of the different proteins to which it binds, the ambiguity can be resolved by decoding the binding profiles for each protein using machine learning or artificial intelligence algorithms that are based on probabilities for the affinity reagents binding to candidate proteins. For example, a plurality of different promiscuous affinity reagents can be contacted with a complex population of proteins, wherein the plurality is configured to produce a different binding profile for each candidate protein suspected of being present in the population. The plurality of promiscuous affinity reagents can produce a binding profile for each individual protein that can be decoded to identify a unique combination of positive binding outcomes (i.e. observed binding events) and/or negative binding outcomes (i.e. observed non-binding events), and this can in turn be used to identify the individual protein as a particular candidate protein having a high likelihood of exhibiting a similar binding profile.

Binding profiles can be obtained for sample proteins and/or epitope display proteins and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include information regarding the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an unknown protein in a sample, an empirical binding profile for the protein can be compared to results computed by the binding model for many or all candidate proteins suspected of being in the sample. A machine learning or artificial intelligence algorithm can be used. An algorithm used for decoding can utilize Bayesian inference. In some configurations, identity of an unknown protein is determined based on a likelihood of the unknown protein being a particular candidate protein given the empirical binding pattern or based on the probability of a particular candidate protein generating the empirical binding pattern. Particularly useful decoding methods are set forth, for example, in U.S. Pat. No. 10,473,654; U.S. Pat. App. Pub. No. 2020/0318101 A1; U.S. patent application Ser. No. 18/045,036, or Egertson et al., BioRxiv (2021), DOI: 10.1101/2021.10.11.463967, each of which is incorporated herein by reference. A method of the present disclosure can be configured to identify at least one sample protein from an organism based on known identity, or determined identity, of at least one epitope display proteins. For example, results of decoding a sample protein can be compared to results of decoding an epitope display protein.

One or more compositions set forth herein can be provided in kit form including, if desired, a suitable packaging material. In one configuration, for example, a particle, solid support, flow cell, array, epitope display protein, affinity reagent, assay reagent and/or other composition set forth herein can be provided in one or more vessels. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

Accordingly, the present disclosure provides a kit including an epitope display protein and an affinity reagent that recognizes an epitope of the epitope display protein. For example, a kit can include an epitope display protein listed in Table 1 or Table 2. Optionally, a kit can include (a) a protein, comprising an amino acid sequence that is at least 80% identical to EDP1, wherein X₁, X₂, X₃, X₄ and X₅ each include a sequence of at least 2 amino acids and at most 10 amino acids; and (b) an affinity reagent that recognizes an epitope present in X₁, X₂, X₃, X₄ or X₅. Optionally, a kit can include (a) a protein, comprising an amino acid sequence that is at least 80% identical to EDP2, wherein X₁, X₂, X₃, X₄, X₅, X₆, X₇, X₈, X₉, and X₁₀ each comprise a sequence of at least 2 amino acids and at most 10 amino acids; and (b) an affinity reagent that recognizes an epitope present in X₁, X₂, X₃, X₄, X₅, X₆, X₇, X₈, X₉, or X₁₀.

### Example I

### Design of the EDP1 Epitope Display Protein

The Peak6 protein was identified as a candidate for design of an epitope display protein based on several favorable characteristics. For example, Peak6 (1) is a relatively small protein (77 amino acid residues), (2) has a relatively compact structure (3) includes five surface exposed loops, (4) has been successfully expressed in a recombinant system, (5) has been structurally characterized at 1.54 angstrom resolution, and (6) having been de novo designed, is amenable to a priori prediction and characterization with respect to primary, secondary and tertiary structures. See Koepnick et al., Nature 570:390-394 (2019) and PDB DOI: 10.2210/pdb6MRS/pdb, each of which is incorporated herein by reference.

An epitope display protein, pre-GHSPG₅, was designed to include regular secondary structure elements of Peak6 protein, and this epitope display structure motif was fused to a pre-sequence. The pre-sequence included a single cysteine, the cysteine being unique to the epitope display protein, a His-Tag (i.e. 6 sequential histidine residues) and a TEV protease recognition sequence. According to the design treatment of the pre-GHSPG₅ protein with TEV protease will produce the GHSPG₅ protein. The primary sequences of pre-GHSPG₅ and GHSPG₅ are aligned with each other in FIG. 2A along with an alignment to regions of regular secondary structure. The sequence of secondary structures of the epitope display structure motif of pre-GHSPG₅ and GHSPG₅ is alpha₁-beta₁-beta₂-alpha₂-beta₃-beta₄, wherein “alpha” indicates an alpha helix and “beta” indicates a beta strand. The regular secondary structures provide a scaffold for the motif. The motif further includes loop X₁ connecting alpha₁-beta₁, loop X₂ connecting beta₁-beta₂, loop X₃ connecting beta₂-alpha₂, loop X₄ connecting alpha₂-beta₃, and loop X₅ connecting beta₃-beta₄. Loop X₅ of pre-GHSPG₅ and GHSPG₅ are configured to display the HSP timer epitope and the other four loops have the sequences found in Peak6.

The structure for pre-GHSPG₅ was predicted using the alphaFold (DEEPMind Ltd., London UK) module ColabFold (Mirdita et al. Nat Methods. Jun; 19:679-682 (2022), which is incorporated herein by reference) built into the molecular visualization software ChimeraX (Pettersen et al. Protein Sci. 30:70-82 (2021), which is incorporated herein by reference), protein structures were predicted by entering the sequence of the primary structure.

### Example II

### Manufacture and Characterization of Epitope Display Proteins Having the EDP1 Epitope Display Structure Motif

The pre-GHSPG₅ protein was cloned and expressed as follows. The pET-29b (+) expression vector, containing the gene for the preGHSPG protein (Table 3) was ordered from Genscript Biotech (NJ, USA). The vector was transformed into BL21 Star™ (DE3) pLysS One Shot™ chemically competent cells (Thermo Fischer Scientific) following manufacturer's recommendation onto LB agar plates containing 50 μg/mL kanamycin and 34 μg/mL chloramphenicol. Single colonies were picked and grown in 5 mL Luria Broth (Teknova) containing 50 μg/mL kanamycin and 34 μg/mL chloramphenicol overnight at 37° C. shaking at 225 rpm. The following day, the 5 mL starter culture was added to 1 L Luria broth containing 50 μg/mL kanamycin and 34 μg/mL chloramphenicol and incubated at 37° C. shaking at 225 rpm until the optical density of the culture reached 0.6. Isopropyl β-D-1-thiogalactopyranoside (Fisher Scientific) was added to the culture at a final concentration of 1 mM, the temperature was reduced to 25° C. and grown overnight shaking at 225 rpm.

The pre-GHSPG₅ protein was purified and processed as follows. Cells were harvested by centrifugation at 4000 rpm for 10 minutes. Cells were resuspended in lysis buffer containing 20 mM TRIS pH 7.4, 300 mM sodium chloride, 1 mM phenylmethanesulfonyl fluoride (Roche) and 1 mg/mL lysozyme (Sigma) and frozen in liquid nitrogen. Cells were then thawed in warm water and sonicated on ice with stirring using a Qsonic Q125 tip sonicator equipped with a 3.2 mm tip at 50% amplitude with a 30 secs on/30 secs off pulse pattern for 5 minutes. Samples were then filtered through a 0.22 μm syringe filter and mixed with 5 mL NEBExpress® Ni Resin (New England Biolabs) and incubated on a rotator at 4° C. for 30 minutes. Samples were transferred to a gravity purification column and resin was allowed to settle while lysis buffer was removed. The column was washed with 50 mL of wash buffer containing 20 mM TRIS pH 7.4, 300 mM sodium chloride, and 30 mM imidazole. Samples were eluted in 5 mL elution buffer containing 20 mM TRIS pH 7.4, 300 mM sodium chloride, and 250 mM imidazole. Samples were dialyzed into storage buffer contain 10 mM HEPES pH 7.5, 50 mM sodium chloride, 2.5 mM 2-mercaptoethanol, and 15% glycerol (v/v).

The GHSPG₅ protein was characterized using the following assay. The protein was biotinylated through lysine residues using NHS-Biotin. The protein was pulled down using streptavidin magnetic beads. An antibody was incubated with the bead immobilized protein, and excess antibody was washed away. Finally, the antibody was detected using an alexa647-labeled anti-human IgG secondary antibody and fluorescence intensity was read. The assay was carried out for three samples including (1) antibody 19328, which was selected to recognize the DPY epitope; (2) antibody 19316, which was selected to recognize the HSP epitope; and (3) no antibody, negative control.

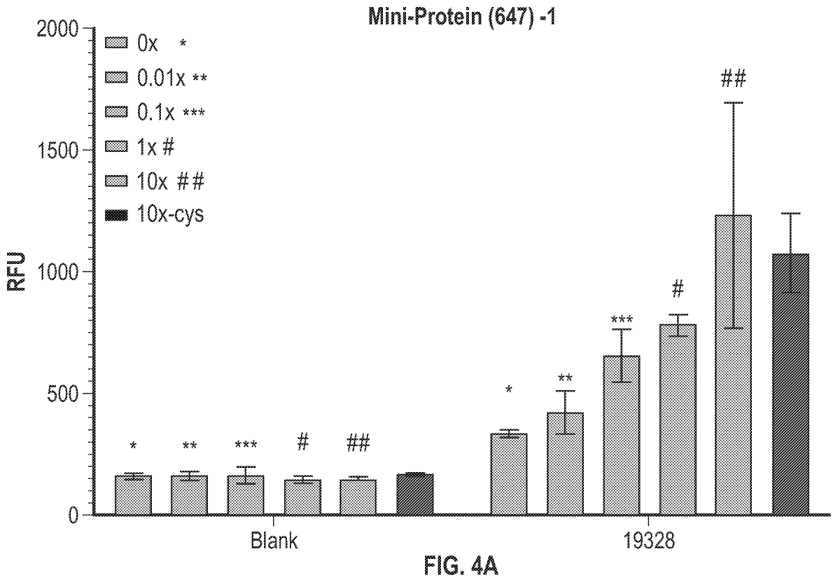

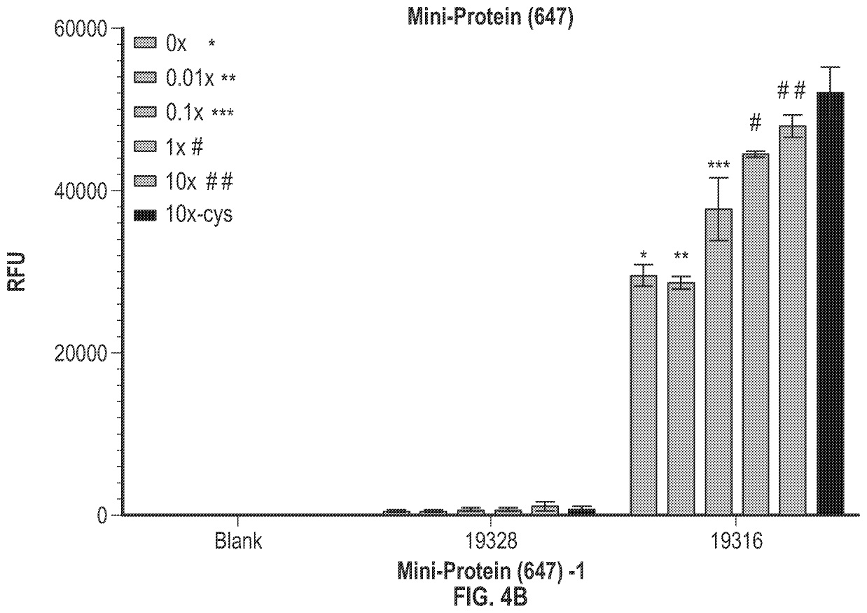

Results of the assay are shown in FIG. 4A and FIG. 4B. FIG. 4A shows data for binding of the GHSPG₅ protein (identified as “mini-protein 647” in the figure) to various concentrations of antibodies 19328 and 19316, and negative controls having no antibodies are also shown (blank). FIG. 4B shows the same data for antibody 19328 and the negative control; however, the y-axis is rescaled. Antibody concentrations listed top to bottom in the legend correspond to positions from left to right, respectively, on the x-axis for each antibody.

TABLE 3
Nucleotide Sequence Encoding the preGHSPG Protein

Gene: preGHSPG
Sequence: SEQ ID NO: 76
AGACGGGCAACAGCTGATTGCCCTTCACCGCCTGGCCCTGAGAGAGTTG
CAGCAAGCGGTCCACGCTGGTTTGCCCCAGCAGGCGAAAATCCTGTTTG
ATGGTGGTTAACGGCGGGATATAACATGAGCTGTCTTCGGTATCGTCGT
ATCCCACTACCGAGATGTCCGCACCAACGCGCAGCCCGGACTCGGTAAT
GGCGCGCATTGCGCCCAGCGCCATCTGATCGTTGGCAACCAGCATCGCA
GTGGGAACGATGCCCTCATTCAGCATTTGCATGGTTTGTTGAAAACCGG
ACATGGCACTCCAGTCGCCTTCCCGTTCCGCTATCGGCTGAATTTGATT
GCGAGTGAGATATTTATGCCAGCCAGCCAGACGCAGACGCGCCGAGACA
GAACTTAATGGGCCCGCTAACAGCGCGATTTGCTGGTGACCCAATGCGA
CCAGATGCTCCACGCCCAGTCGCGTACCGTCTTCATGGGAGAAAATAAT
ACTGTTGATGGGTGTCTGGTCAGAGACATCAAGAAATAACGCCGGAACA
TTAGTGCAGGCAGCTTCCACAGCAATGGCATCCTGGTCATCCAGCGGAT
AGTTAATGATCAGCCCACTGACGCGTTGCGCGAGAAGATTGTGCACCGC
CGCTTTACAGGCTTCGACGCCGCTTCGTTCTACCATCGACACCACCACG
CTGGCACCCAGTTGATCGGCGCGAGATTTAATCGCCGCGACAATTTGCG
ACGGCGCGTGCAGGGCCAGACTGGAGGTGGCAACGCCAATCAGCAACGA
CTGTTTGCCCGCCAGTTGTTGTGCCACGCGGTTGGGAATGTAATTCAGC
TCCGCCATCGCCGCTTCCACTTTTTCCCGCGTTTTCGCAGAAACGTGGC
TGGCCTGGTTCACCACGCGGGAAACGGTCTGATAAGAGACACCGGCATA
CTCTGCGACATCGTATAACGTTACTGGTTTCACATTCACCACCCTGAAT
TGACTCTCTTCCGGGCGCTATCATGCCATACCGCGAAAGGTTTTGCGCC
ATTCGATGGTGTCCGGGATCTCGACGCTCTCCCTTATGCGACTCCTGCA
TTAGGAAGCAGCCCAGTAGTAGGTTGAGGCCGTTGAGCACCGCCGCCGC
AAGGAATGGTGCATGCAAGGAGATGGCGCCCAACAGTCCCCCGGCCACG
GGGCCTGCCACCATACCCACGCCGAAACAAGCGCTCATGAGCCCGAAGT
GGCGAGCCCGATCTTCCCCATCGGTGATGTCGGCGATATAGGCGCCAGC
AACCGCACCTGTGGCGCCGGTGATGCCGGCCACGATGCGTCCGGCGTAG
AGGATCGAGATCGATCTCGATCCCGCGAAATTAATACGACTCACTATAG
GGGAATTGTGAGCGGATAACAATTCCCCTCTAGAAATAATTTTGTTTAA
CTTTAAGAAGGAGATATACATATGTGTGGGCACCACCACCATCACCATG
GATGGTCTGAAAACCTGTACTTCCAGGGCAGCGGCCGTCAGGAGAAAGT
TCTGAAGTCCATCGAGGAGACTGTACGCAAAATGGGTGTTACCATGGAA
ACCCATCGTAGCGGTAATGAAGTTAAAGTGGTGATCAAGGGTCTGCACG
AGTCGCAACAAGAGCAGTTGAAGAAGGACGTTGAAGAGACGAGCAAAAA
GCAAGGCGTCGAGACCCGTATTGAATTTGGTCACAGCCCGGGCACCGTG
ACCATTGTCGTGCGCGAATAACTCGAGCACCACCACCACCACCACTGAG
ATCCGGCTGCTAACAAAGCCCGAAAGGAAGCTGAGTTGGCTGCTGCCAC
CGCTGAGCAATAACTAGCATAACCCCTTGGGGCCTCTAAACGGGTCTTG
AGGGGTTTTTTGCTGAAAGGAGGAACTATATCCGGATCTCGAG

What is claimed is:

1. A protein, comprising an amino acid sequence that is at least 80% identical to GSGRQEKVLKSIEETVX₁ETHX₂VKVVX₃ESQQEQLKKDVEETSKKQX₄RIEFX₅VTIVVRE (SEQ ID NO: 2); wherein X₁, X₂, X₃, X₄ and X₅ each comprise a sequence of at least 2 amino acids and at most 10 amino acids; and wherein X₁ is not RKMGVTM, X₂ is not RSGNE, X₃ is not IKGLH, X₄ is not GVET, or X₅ is not HGDT.

2. The protein of claim 1, wherein at least two of X₁, X₂, X₃, X₄ and X₅ each comprises an identical sequence of three to six amino acids.

3. The protein of claim 2, wherein X₁, X₂, X₃, X₄ and X₅ each comprises an identical sequence of three to six amino acids.

4. The protein of claim 1, wherein X₁, X₂, X₃, X₄ and X₅ comprise different amino acid sequences.

5. The protein of claim 1, wherein the amino acid sequence comprises GSGRQEKVLKSIEETVX₁ETHX₂VKVVX₃ESQQEQLKKDVEETSKKQX₄RIEFXVTIVVRE (SEQ ID NO: 2).

6. The protein of claim 1, wherein the amino acid sequence forms a series of secondary structures comprising alpha₁-X₁-beta₁-X₂-beta₂-X₃-alpha₂-X₄-beta₃-X₅-beta₄, wherein alpha₁ and alpha₂ each comprises an alpha helix, and wherein beta₁, beta₂, beta₃, and beta₄ each comprises a beta strand.

7. The protein of claim 1, wherein X₁, X₂, X₃, X₄ and X₅ each comprises an irregular secondary structure.

8. The protein of claim 1, comprising a tertiary structure wherein X₁, X₂, X₃, X₄ and X₅ each comprises a solvent exposed loop region.

9. The protein of claim 1, comprising a tertiary structure having a template modeling score of at least 0.5 when compared to the tertiary structure formed by the amino acid sequence GSGRQEKVLKSIEETVRKMGVTMETHRSGNEVKVVIKGLHESQQEQLKKDVEETSKKQ GVETRIEFHGDTVTIVVRE (SEQ ID NO: 1).

10. The protein of claim 1, wherein a single cysteine is present in the protein.

11. The protein of claim 1, further comprising an additional N-terminal sequence region comprising MCGHHHHHHGWSENLYFQ (SEQ ID NO: 73).

12. The protein of claim 1, wherein an affinity reagent is non-covalently bound to X₁, X₂, X₃, X₄ or X₅.

13. The protein of claim 12, wherein the affinity reagent comprises an antibody or nucleic acid aptamer.

14. The protein of claim 1, wherein X₁ comprises an amino acid sequence selected from the group consisting of HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

15. The protein of claim 1, wherein X₂ comprises an amino acid sequence selected from the group consisting of HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

16. The protein of claim 1, wherein X₃ comprises an amino acid sequence selected from the group consisting of HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

17. The protein of claim 1, wherein X₄ comprises an amino acid sequence selected from the group consisting of HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

18. The protein of claim 1, wherein X₅ comprises an amino acid sequence selected from the group consisting of HHH, HRH, YFR, WNK, FRRF, RFRF, WFR, LEEL, YWL, HFR, FST, DPY, FWR, DTR, DTV, RWWR, RDE, HSP, DPY, DTR, SLF, and DDY.

19. A solid support comprising the protein of claim 1 attached to the solid support.

20. The solid support of claim 19, wherein an array of different proteins is attached to the solid support.


SEQUENCE LISTING (76 sequences)

SEQ ID NO: 1 - protein, 77 aa, synthetic construct
GSGRQEKVLKSIEETVRKMGVTMETHRSGNEVKVVIKGLHESQQEQLKKDVEETSKKQGVETRIEFHGDTVTIVVRE

SEQ ID NO: 2 - protein, 102 aa, synthetic construct
  VARIANT 17..26: note = one to eight X residues may be absent
  VARIANT 30..39: note = one to eight X residues may be absent
  VARIANT 44..53: note = one to eight X residues may be absent
  VARIANT 72..81: note = one to eight X residues may be absent
  VARIANT 86..95: note = one to eight X residues may be absent
GSGRQEKVLKSIEETVXXXXXXXXXXETHXXXXXXXXXXVKVVXXXXXXXXXXESQQEQLKKDVEETSKKQXXXXXXXXXXRIEFXXXXXXXXXXVTIVVRE

SEQ ID NO: 3 - protein, 119 aa, synthetic construct
  VARIANT 34..43: note = 1 to 8 X residues may be absent
  VARIANT 47..56: note = one to eight X residues may be absent
  VARIANT 61..70: note = one to eight X residues may be absent
  VARIANT 89..98: note = one to eight X residues may be absent
  VARIANT 103..112: note = one to eight X residues may be absent
MGHHHHHHGWSENLYFQGSGRQEKVLKSIEETVXXXXXXXXXXETHXXXXXXXXXXVKVVXXXXXXXXXXESQQEQLKKDVEETSKKQXXXXXXXXXXRIEFXXXXXXXXXXVTIVVRE

SEQ ID NO: 4 - protein, 80 aa, synthetic construct
  VARIANT 17..26: note = one to eight X residues may be absent
GSGRQEKVLKSIEETVXXXXXXXXXXETHRSGNEVKVVIKGLHESQQEQLKKDVEETSKKQGVETRIEFHGDTVTIVVRE

SEQ ID NO: 5 - protein, 97 aa, synthetic construct
  VARIANT 34..43: note = one to eight X residues may be absent
MGHHHHHHGWSENLYFQGSGRQEKVLKSIEETVXXXXXXXXXXETHRSGNEVKVVIKGLHESQQEQLKKDVEETSKKQGVETRIEFHGDTVTIVVRE

SEQ ID NO: 6 - protein, 82 aa, synthetic construct
  VARIANT 27..36: note = one to eight X residues may be absent
GSGRQEKVLKSIEETVRKMGVTMETHXXXXXXXXXXVKVVIKGLHESQQEQLKKDVEETSKKQGVETRIEFHGDTVTIVVRE

SEQ ID NO: 7 - protein, 99 aa, synthetic construct
  VARIANT 44..53: note = one to eight X residues may be absent
MGHHHHHHGWSENLYFQGSGRQEKVLKSIEETVRKMGVTMETHXXXXXXXXXXVKVVIKGLHESQQEQLKKDVEETSKKQGVETRIEFHGDTVTIVVRE

SEQ ID NO: 8 - protein, 82 aa, synthetic construct
  VARIANT 36..45: note = one to eight X residues may be absent
GSGRQEKVLKSIEETVRKMGVTMETHRSGNEVKVVXXXXXXXXXXESQQEQLKKDVEETSKKQGVETRIEFHGDTVTIVVRE

SEQ ID NO: 9 - protein, 99 aa, synthetic construct
  VARIANT 53..62: note = one to eight X residues may be absent
MGHHHHHHGWSENLYFQGSGRQEKVLKSIEETVRKMGVTMETHRSGNEVKVVXXXXXXXXXXESQQEQLKKDVEETSKKQGVETRIEFHGDTVTIVVRE

SEQ ID NO: 10 - protein, 83 aa, synthetic construct
  VARIANT 59..68: note = one to eight X residues may be absent
GSGRQEKVLKSIEETVRKMGVTMETHRSGNEVKVVIKGLHESQQEQLKKDVEETSKKQXXXXXXXXXXRIEFHGDTVTIVVRE

SEQ ID NO: 11 - protein, 100 aa, synthetic construct
  VARIANT 76..85: note = one to eight X residues may be absent
MGHHHHHHGWSENLYFQGSGRQEKVLKSIEETVRKMGVTMETHRSGNEVKVVIKGLHESQQEQLKKDVEETSKKQXXXXXXXXXXRIEFHGDTVTIVVRE

SEQ ID NO: 12 - protein, 83 aa, synthetic construct
  VARIANT 67..76: note = one to eight X residues may be absent
GSGRQEKVLKSIEETVRKMGVTMETHRSGNEVKVVIKGLHESQQEQLKKDVEETSKKQGVETRIEFXXXXXXXXXXVTIVVRE

SEQ ID NO: 13 - protein, 100 aa, synthetic construct
  VARIANT 84..93: note = one to eight X residues may be absent
MGHHHHHHGWSENLYFQGSGRQEKVLKSIEETVRKMGVTMETHRSGNEVKVVIKGLHESQQEQLKKDVEETSKKQGVETRIEFXXXXXXXXXXVTIVVRE

SEQ ID NO: 14 - protein, 79 aa, synthetic construct
GSGRQEKVLKSIEETVRKMGVTMETHRSGNEVKVVIKGLHESQQEQLKKDVEETSKKQGVETRIEFGHSPGTVTIVVRE

SEQ ID NO: 15 - protein, 97 aa, synthetic construct
MCGHHHHHHGWSENLYFQGSGRQEKVLKSIEETVRKMGVTMETHRSGNEVKVVIKGLHESQQEQLKKDVEETSKKQGVETRIEFGHSPGTVTIVVRE

SEQ ID NO: 16 - protein, 79 aa, synthetic construct
GSGRQEKVLKSIEETVRKMGVTMETHRSGNEVKVVIKGLHESQQEQLKKDVEETSKKQGVETRIEFGDPYGTVTIVVRE

SEQ ID NO: 17 - protein, 97 aa, synthetic construct
MCGHHHHHHGWSENLYFQGSGRQEKVLKSIEETVRKMGVTMETHRSGNEVKVVIKGLHESQQEQLKKDVEETSKKQGVETRIEFGDPYGTVTIVVRE

SEQ ID NO: 18 - protein, 79 aa, synthetic construct
GSGRQEKVLKSIEETVRKMGVTMETHRSGNEVKVVIKGLHESQQEQLKKDVEETSKKQGVETRIEFGWNKGTVTIVVRE

SEQ ID NO: 19 - protein, 97 aa, synthetic construct
MCGHHHHHHGWSENLYFQGSGRQEKVLKSIEETVRKMGVTMETHRSGNEVKVVIKGLHESQQEQLKKDVEETSKKQGVETRIEFGWNKGTVTIVVRE

SEQ ID NO: 20 - protein, 80 aa, synthetic construct
GSGRQEKVLKSIEETVRGHSPGMETHRGDPYGVKVVIGWNKGHESQQEQLKKDVEETSKKQGDTRGRIEFHGDTVTIVVR

SEQ ID NO: 21 - protein, 99 aa, synthetic construct
MCGHHHHHHGWSENLYFQGSGRQEKVLKSIEETVRGHSPGMETHRGDPYGVKVVIGWNKGHESQQEQLKKDVEETSKKQGDTRGRIEFHGDTVTIVVRE

SEQ ID NO: 22 - protein, 82 aa, synthetic construct
GSGRQEKVLKSIEETVRKMGDPYGTMETHRSGNEVKVVIKGLHESQQEQLKKDVEETSKKQGVETRIEFGHSPGTVTIVVRE

SEQ ID NO: 23 - protein, 100 aa, synthetic construct
MCGHHHHHHGWSENLYFQGSGRQEKVLKSIEETVRKMGDPYGTMETHRSGNEVKVVIKGLHESQQEQLKKDVEETSKKQGVETRIEFGHSPGTVTIVVRE

SEQ ID NO: 24 - protein, 81 aa, synthetic construct
GSGRQEKVLKSIEETVRKMGDPYGTMETHGSLFGVKVVIKGLHESQQEQLKKDVEETSKKQGVETRIEFGHSPGTVTIVVR

SEQ ID NO: 25 - protein, 100 aa, synthetic construct
MCGHHHHHHGWSENLYFQGSGRQEKVLKSIEETVRKMGDPYGTMETHGSLFGVKVVIKGLHESQQEQLKKDVEETSKKQGVETRIEFGHSPGTVTIVVRE

SEQ ID NO: 26 - protein, 83 aa, synthetic construct
GSGRQEKVLKSIEETVRKMGDPYGTMETHGSLFGVKVVIKGLHESQQEQLKKDVEETSKKQGRDEGRIEFGHSPGTVTIVVRE

SEQ ID NO: 27 - protein, 101 aa, synthetic construct
MCGHHHHHHGWSENLYFQGSGRQEKVLKSIEETVRKMGDPYGTMETHGSLFGVKVVIKGLHESQQEQLKKDVEETSKKQGRDEGRIEFGHSPGTVTIVVRE

SEQ ID NO: 28 - protein, 83 aa, synthetic construct
GSGRQEKVLKSIEETVRKMGDPYGTMETHGSLFGVKVVGDDYGESQQEQLKKDVEETSKKQGRDEGRIEFGHSPGTVTIVVRE

SEQ ID NO: 29 - protein, 101 aa, synthetic construct
MCGHHHHHHGWSENLYFQGSGRQEKVLKSIEETVRKMGDPYGTMETHGSLFGVKVVGDDYGESQQEQLKKDVEETSKKQGRDEGRIEFGHSPGTVTIVVRE

SEQ ID NO: 30 - protein, 77 aa, synthetic construct
GSGRQEKVLKSIEETVRKMGVTMETHRSGNEVKVVIKGLHESQQEHSPKDVEETSKKQGVETRIEFHGDTVTIVVRE

SEQ ID NO: 31 - protein, 94 aa, synthetic construct
MGHHHHHHGWSENLYFQGSGRQEKVLKSIEETVRKMGVTMETHRSGNEVKVVIKGLHESQQEHSPKDVEETSKKQGVETRIEFHGDTVTIVVRE

SEQ ID NO: 32 - protein, 4 aa, synthetic construct
FRRF

SEQ ID NO: 33 - protein, 4 aa, synthetic construct
RFRF

SEQ ID NO: 34 - protein, 4 aa, synthetic construct
LEEL

SEQ ID NO: 35 - protein, 4 aa, synthetic construct
RWWR

SEQ ID NO: 36 - protein, 7 aa, synthetic construct
RKMGVTM

SEQ ID NO: 37 - protein, 5 aa, synthetic construct
RSGNE

SEQ ID NO: 38 - protein, 5 aa, synthetic construct
IKGLH

SEQ ID NO: 39 - protein, 4 aa, synthetic construct
GVET

SEQ ID NO: 40 - protein, 4 aa, synthetic construct
HGDT

SEQ ID NO: 41 - protein, 7 aa, synthetic construct
RGHSPGM

SEQ ID NO: 42 - protein, 5 aa, synthetic construct
GHSPG

SEQ ID NO: 43 - protein, 5 aa, synthetic construct
GDPYG

SEQ ID NO: 44 - protein, 5 aa, synthetic construct
GWNKG

SEQ ID NO: 45-49 - skipped (5 entries)

SEQ ID NO: 50 - protein, 283 aa, synthetic construct
EESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 51 - protein, 327 aa, synthetic construct
  VARIANT 22..31: note = one to eight X residues may be absent
  VARIANT 40..49: note = one to eight X residues may be absent
  VARIANT 80..89: note = one to eight X residues may be absent
  VARIANT 100..109: note = one to eight X residues may be absent
  VARIANT 131..140: note = one to eight X residues may be absent
  VARIANT 176..185: note = one to eight X residues may be absent
  VARIANT 198..207: note = one to eight X residues may be absent
  VARIANT 217..226: note = one to eight X residues may be absent
  VARIANT 249..258: note = one to eight X residues may be absent
  VARIANT 268..277: note = one to eight X residues may be absent
MESKKRQWALEDFEIGRPLGKXXXXXXXXXXGNVYLAREXXXXXXXXXXILALKVLFKAQLEKAGVEHQLRREVEIQSHXXXXXXXXXXNILRLYGYFHXXXXXXXXXXRVYLILEYAPLGTVYRELQKLXXXXXXXXXXEQRTATYITELANALSYCHSKRVIHRDIKPENLLLXXXXXXXXXXLKIADFGWSVHAXXXXXXXXXXLDYLPPEMIXXXXXXXXXXEKVDLWSLGVLCYEFLVGKPPFXXXXXXXXXXYQETYKRISXXXXXXXXXXEGARDLISRLLKHNPSQRPMLREVLEHPWITANSSKPSNAQNKESASKQS

SEQ ID NO: 52 - protein, 610 aa, synthetic construct
  VARIANT 289..298: note = one to eight X residues may be absent
  VARIANT 307..316: note = one to eight X residues may be absent
  VARIANT 347..356: note = one to eight X residues may be absent
  VARIANT 367..376: note = one to eight X residues may be absent
  VARIANT 398..407: note = one to eight X residues may be absent
  VARIANT 443..452: note = one to eight X residues may be absent
  VARIANT 465..474: note = one to eight X residues may be absent
  VARIANT 484..493: note = one to eight X residues may be absent
  VARIANT 516..525: note = one to eight X residues may be absent
  VARIANT 535..544: note = one to eight X residues may be absent
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTMESKKRQWALEDFEIGRPLGKXXXXXXXXXXGNVYLAREXXXXXXXXXXILALKVLFKAQLEKAGVEHQLRREVEIQSHXXXXXXXXXXNILRLYGYFHXXXXXXXXXXRVYLILEYAPLGTVYRELQKLXXXXXXXXXXEQRTATYITELANALSYCHSKRVIHRDIKPENLLLXXXXXXXXXXLKIADFGWSVHAXXXXXXXXXXLDYLPPEMIXXXXXXXXXXEKVDLWSLGVLCYEFLVGKPPFXXXXXXXXXXYQETYKRISXXXXXXXXXXEGARDLISRLLKHNPSQRPMLREVLEHPWITANSSKPSNAQNKESASKQSDYKDDDDKHHHHHHHH

SEQ ID NO: 53 - protein, 283 aa, synthetic construct
MESKKRQWALEDFEIGRPLGKSLFGNVYLAREKDTRFILALKVLFKAQLEKAGVEHQLRREVEIQSHLLPQNILRLYGYFHLEFRVYLILEYAPLGTVYRELQKLHSPDEQRTATYITELANALSYCHSKRVIHRDIKPENLLLGHPDELKIADFGWSVHAPSSRRDRIAGTLDYLPPEMIEGRFSTEKVDLWSLGVLCYEFLVGKPPFFRETYQETYKRISRVEFTFPSVHTEGARDLISRLLKHNPSQRPMLREVLEHPWITANSSKPSNAQNKESASKQS

SEQ ID NO: 54 - protein, 566 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTMESKKRQWALEDFEIGRPLGKSLFGNVYLAREKDTRFILALKVLFKAQLEKAGVEHQLRREVEIQSHLLPQNILRLYGYFHLEFRVYLILEYAPLGTVYRELQKLHSPDEQRTATYITELANALSYCHSKRVIHRDIKPENLLLGHPDELKIADFGWSVHAPSSRRDRIAGTLDYLPPEMIEGRFSTEKVDLWSLGVLCYEFLVGKPPFFRETYQETYKRISRVEFTFPSVHTEGARDLISRLLKHNPSQRPMLREVLEHPWITANSSKPSNAQNKESASKQSDYKDDDDKHHHHHHHH

SEQ ID NO: 55 - protein, 290 aa, synthetic construct
  VARIANT 22..31: note = one to eight X residues may be absent
MESKKRQWALEDFEIGRPLGKXXXXXXXXXXGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 56 - protein, 288 aa, synthetic construct
  VARIANT 33..42: note = one to eight X residues may be absent
MESKKRQWALEDFEIGRPLGKGKFGNVYLAREXXXXXXXXXXILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 57 - protein, 289 aa, synthetic construct
  VARIANT 68..77: note = one to eight X residues may be absent
MESKKRQWALEDFEIGRPLGKGKFGNVYLAREKQSKFILALKVLFKAQLEKAGVEHQLRREVEIQSHXXXXXXXXXXNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 58 - protein, 290 aa, synthetic construct
  VARIANT 82..91: note = one to eight X residues may be absent
MESKKRQWALEDFEIGRPLGKGKFGNVYLAREKQSKFILALKVLFKAQLEKAGVEHQLRREVEIQSHLRHPNILRLYGYFHXXXXXXXXXXRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 59 - protein, 289 aa, synthetic construct
  VARIANT 106..115: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLXXXXXXXXXXEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 60 - protein, 288 aa, synthetic construct
  VARIANT 145..154: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLXXXXXXXXXXLKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 61 - protein, 282 aa, synthetic construct
  VARIANT 162..171: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAXXXXXXXXXXLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 62 - protein, 287 aa, synthetic construct
  VARIANT 182..191: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIXXXXXXXXXXEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 63 - protein, 289 aa, synthetic construct
  VARIANT 210..219: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFXXXXXXXXXXYQETYKRISRVEFTFPDFVTEGARDLISRLLKHNPSQRPMLREVLEHPWITANSSKPSNCQNKESASKQS

SEQ ID NO: 64 - protein, 282 aa, synthetic construct
  VARIANT 223..232: note = one to eight X residues may be absent
MESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISXXXXXXXXXXEGARDLISRLLKHNPSQRPMLREVLEHPWITANSSKPSNCQNKESASKQS

SEQ ID NO: 65 - protein, 5 aa, synthetic construct
KQSKF

SEQ ID NO: 66 - protein, 4 aa, synthetic construct
LRHP

SEQ ID NO: 67 - protein, 4 aa, synthetic construct
SKFD

SEQ ID NO: 68 - protein, 5 aa, synthetic construct
GSAGE

SEQ ID NO: 69 - protein, 11 aa, synthetic construct
PSSRRTTLCGT

SEQ ID NO: 70 - protein, 6 aa, synthetic construct
EGRMHD

SEQ ID NO: 71 - protein, 4 aa, synthetic construct
EANT

SEQ ID NO: 72 - protein, 11 aa, synthetic construct
RVEFTFPDFVT

SEQ ID NO: 73 - protein, 18 aa, synthetic construct
MCGHHHHHHGWSENLYFQ

SEQ ID NO: 74 - protein, 267 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGT

SEQ ID NO: 75 - protein, 6 aa, synthetic construct
LVPRGS

SEQ ID NO: 76 - DNA, 1905 bp, synthetic construct
  source 1..1905: mol_type = other DNA
agacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgtgtgggcaccaccaccatcaccatggatggtctgaaaacctgtacttccagggcagcggccgtcaggagaaagttctgaagtccatcgaggagactgtacgcaaaatgggtgttaccatggaaacccatcgtagcggtaatgaagttaaagtggtgatcaagggtctgcacgagtcgcaacaagagcagttgaagaaggacgttgaagagacgagcaaaaagcaaggcgtcgagacccgtattgaatttggtcacagcccgggcaccgtgaccattgtcgtgcgcgaataactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggatctcgag